(19) United States
(12) Patent Application Publication
(10) Pub. No.: US 2025/0312485 A1
(43) Pub. Date: Oct. 9, 2025

(54) COMPOSITIONS AND METHODS FOR THE MANAGEMENT AND TREATMENT OF PHENYLKETONURIA

(71) Applicant: THE TRUSTEES OF THE UNIVERSITY OF PENNSYLVANIA, Philadelphia, PA (US)

(72) Inventors: Kiran MUSUNURU, Philadelphia, PA (US); Xiao WANG, Merion Station, PA (US); Dominique Lynnette BROOKS, Philadelphia, PA (US)

(73) Assignee: THE TRUSTEES OF THE UNIVERSITY OF PENNSYLVANIA, Philadelphia, PA (US)

(21) Appl. No.: 18/854,319

(22) PCT Filed: Apr. 7, 2023
(86) PCT No.: PCT/US2023/065536

§ 371 Date: Oct. 4, 2024

Related U.S. Application Data
(60) Provisional application No. 63/328,492, filed on Apr. 7, 2022.

(51) Int. Cl. A61K 48/00; A01K 67/0278; A61K 9/1272; A61K 9/51; A61K 38/44; A61P 3/00; C12N 5/071; C12N 9/22; C12N 9/78; C12N 15/11; C12N 15/86; C12N 15/88

(52) U.S. Cl. CPC A61K 48/005; A01K 67/0278; A61K 9/1272; A61K 9/5123; A61K 38/44; A61P 3/00; C12N 5/067; C12N 9/226; C12N 9/78; C12N 15/111; C12N 15/86; C12N 15/88; C12Y 114/16001; A01K 2217/072; A01K 2227/105; C07K 2319/80; C12N 2310/20; C12N 2320/32; C12N 2750/14123; C12N 2750/14143; C12Y 305/04004

(57) ABSTRACT

Compositions and methods for effecting base editing to correct mutations in the phenylalanine hydroxylase gene, thereby curing phenylketonuria, are disclosed.

### CROSS-REFERENCE TO RELATED APPLICATION

This application is a § 371 of International Application No. PCT/US2023/065536, filed Apr. 7, 2023, which claims priority to U.S. Provisional Patent No. 63/328,492 filed Apr. 7, 2022, the entire contents of each being incorporated herein by reference as though set forth in full.

### GRANT SUPPORT STATEMENT

This invention was made with government support under R35HL145203 and R01HL148769 awarded by the National Institutes of Health. The government has certain rights in the invention.

### FIELD OF THE INVENTION

This invention relates to the fields of genetic engineering and correction of genetic errors using base editing therapy. More specifically, the invention provides compositions and methods for correcting gene mutations which cause phenylketonuria.

### INCORPORATION BY REFERENCE OF MATERIAL SUBMITTED IN ELECTRONIC FORM

The Contents of the electronic sequence listing (UPNK-111-US.xml; Size: 425,824 bytes; and Date of Creation: Jun. 23, 2025) is herein incorporated by reference in its entirety.

### BACKGROUND OF THE INVENTION

Several publications and patent documents are cited throughout the specification in order to describe the state of the art to which this invention pertains. Each of these citations is incorporated herein by reference as though set forth in full.

Phenylketonuria (PKU) is an autosomal recessive inborn error of metabolism caused by a deficiency in the hepatic enzyme phenylalanine hydroxylase (PAH). If left untreated, the main clinical feature is intellectual disability. Treatment, which includes a low phenylalanine diet supplemented with amino acid formulas, commences soon after diagnosis within the first weeks of life. Although dietary treatment has been successful in preventing intellectual disability in early treated PKU patients, there are major issues with dietary compliance due to palatability of the diet. Other potential issues associated with dietary therapy include nutritional deficiencies particularly in vitamins D and B12. Suboptimal outcomes in cognitive and executive functioning have been reported in patients who adhere poorly to dietary therapy.

Other approaches include administration of oral medication, e.g., sapropterin, a cofactor of PAH, and an injectable enzyme substitution therapy (pegvaliase). Many PKU patients have limited responses to, or limited access to the medical therapies and, as a result, have impaired cognitive development and develop a range of neuropsychiatric problems. Durable and, ideally, curative therapies are needed to address the unmet medical needs of PKU patients. Although the liver is spared from toxicity, the PAH gene is largely expressed in hepatocytes, and correction of the primary genetic defect solely within the liver would in principle be curative in PKU patients.

It is an object of the invention to provide an effective and lasting treatment of PKU which reduces or eliminates PKU symptoms.

### SUMMARY OF THE INVENTION

The present invention provides compositions and methods for effecting a durable cure of a subset of patients with phenylketonuria via the direct correction of causative mutations for this disease, particularly the c.842C>T mutation, also known as p.Pro281Leu mutation, which is one of the five most common mutations associated with PKU.

In accordance with one aspect of the invention a method for editing a phenylalanine hydroxylase (PAH) encoding polynucleotide comprising mutation associated with phenylketonuria (PKU) is provided. An exemplary method comprises contacting the PAH polynucleotide with a base editor in complex with at least one guide polynucleotide, wherein the base editor comprises a polynucleotide programmable DNA binding domain and an adenosine deaminase domain, and wherein one or more of said guide polynucleotides target said base editor to effect an A⋅T to G⋅C alteration of the mutation associated with PKU thereby restoring the wild-type sequence and correcting the disease phenotype. In certain embodiments, the contacting is done in a cell, a eukaryotic cell, a mammalian cell, or human cell. Contacting may be performed in vitro or in vivo. In particularly preferred embodiments, the mutation is one or more of c. 842C>T (p.Pro281Leu), c. 1222C>T (p.Arg408Trp), c.1066-11G>A, c.782G>A (p.Arg261Gln), c.728G>A (p.Arg243Gln), c.1315+1G>A, and c.473G>A (p.Arg158Gln). The polynucleotide programmable DNA binding domain can be a Streptococcus pyogenes Cas9 (SpCas9) or Staphylococcus aureus Cas9 (SaCas9) or a variant thereof. In certain aspects, the polynucleotide programmable DNA binding domain comprises a modified SpCas9 having an altered protospacer-adjacent motif (PAM) specificity, including without limitation, a modified SpCas9 having specificity for the nucleic acid sequence 5′-NGG-3. The polynucleotide programmable DNA binding domain may be nuclease inactive or nickase variant. In the base editing methods disclosed the adenosine deaminase domain is capable of deaminating adenosine in deoxyribonucleic acid (DNA). The adenosine deaminase can be a TadA deaminase or a variant thereof. In another embodiment, the base editor is in complex with a single guide RNA (sgRNA) comprising a nucleic acid sequence complementary to a nucleic acid sequence comprising the mutation associated with PKU.

Also provided is a cell comprising a base editor, or a polynucleotide encoding said base editor, wherein said base editor comprises a polynucleotide programmable DNA binding domain and an adenosine deaminase domain; and one or more guide polynucleotides that target the base editor to effect an A⋅T to G⋅C alteration of the mutation associated with PKU. In a preferred embodiment, the cell is a hepatocyte obtained from a subject having PKU and expresses PAH polypeptide.

Another embodiment of the invention comprises an adenosine base editor/guide polynucleotide set which corrects a mutation causing PKU. An exemplary set includes (i) a modified SpCas9 or SaCas9; (ii) an adenosine deaminase or functional fragment thereof; and (iii) a guide polynucleotide that targets the base editor to effect an A⋅T to G⋅C alteration of the mutation associated with PKU. In preferred embodiments, the mutation is PAH c.842C>T (p.Pro281Leu) and the guide polynucleotide has a sequence of SEQ ID NO:1 or SEQ ID NO: 3. In other embodiments, the guide polynucleotide comprises a nucleic acid sequence complementary to a PAH encoding nucleic acid sequence of SEQ ID NO:5 or SEQ ID NO:6. The guide polynucleotide can be RNA or DNA or combination thereof.

Another aspect of the invention includes a method of treating PKU in a subject comprising administering to said subject an effective amount the adenosine base editor/guide polynucleotide sets described above. Subjects to be treated include mammals and humans. The base editor, or polynucleotide encoding said base editor, and said one or more guide polynucleotides can be delivered to a cell of the subject, particularly a liver cell.

In one delivery method, the base editor/guide polynucleotide set can be encapsulated in a lipid nanoparticle formulation and delivered to the liver of said subject. In certain aspects, the formulation comprises ionizable cationic lipid, 1,2-distearoyl-sn-glycero-3-phosphocholine, cholesterol, and a PEG-lipid. In an alternative delivery method, the base editor/guide polynucleotide set is delivered to hepatocytes in a single or dual AAV vector system as described herein. In yet another approach, the base editor/guide polynucleotide set can be delivered to hepatocytes in vivo or in vitro in virus-like particles.

### BRIEF DESCRIPTION OF THE DRAWINGS

FIGS. 1A-1C. (FIG. 1A) Adenine base editors (ABEs) are composed of a dead (d) or nickase (n) Cas9 (d/nCas9) fused to one or two TadA, either a TadA evolved to edit adenine in DNA (TadA*) or both a TadA* and a wild-type TadA. ABEs convert A:T into G:C base pairs in the editing window (for example, nucleotide 4 to 7 in the protospacer, in purple). Cas9 is guided by the sgRNA to the protospacer [which is followed by the PAM (protospacer adjacent motif)] and unwinds the DNA, and the deaminase converts the target base. (FIG. 1B) Cytosine base editors (CBEs), composed of a nickase Cas9 (nCas9) fused to a deaminase and one (in BE3s) or two (in BE4s) UGI (uracil glycosylase inhibitor), convert C⋅G into T⋅A base pairs in the editing window (nucleotide 4 to 8 in the protospacer, in green). Undesired events (bystander edits, in blue, and unwanted base conversion, in yellow) of CBEs and ABEs are shown in (FIGS. 1A, 1), respectively. The addition of the second UGI in CBEs (in BE4) and the removal of TadA in ABEs (ABE8) are highlighted with a gray dotted line. The gradient color of the editing window in the upper panels of (FIGS. 1A, 1B) represents the enlarged editing window observed with novel BEs. (FIG. 1C) Successful adenine base editing of PCSK9 in primary human hepatocytes demonstrates proof of principle. Editing of splice-site adenine bases in the PCSK9 gene with 20 gRNAs in hepatocytes transfected with ABE8.8 mRNA and gRNA at three different doses. FIG. 2. Two gRNAs conducive to adenine base editing to correct the PAH c.842C>T variant. This snapshot from the UCSC Browser (SEQ ID NO: 8) shows the site of the PAH c.842C>T (p.Pro281Leu) variant (marked by the vertical yellow bar, with the variant adenine) at the end of the exon. Note that PAH is transcribed in the reverse direction with respect to the reference genome. The target sites for two gRNAs designated PAH1 (SEQ ID NO: 1) and PAH2 (SEQ ID NO: 3) are indicated with a yellow horizontal bar (SEQ ID NO: 9) and a green horizontal bar (SEQ ID NO: 10), respectively. The thick part of each bar corresponds to the protospacer DNA sequence, and the thin part of each bar corresponds to the NGG PAM sequence. The target adenine resulting from the pathogenic G⋅C to A⋅T mutation is in position 5 of the PAH1 protospacer and position 4 of the PAH2 protospacer. The adenine base two positions upstream of the target adenine (black arrow; position 3 of the PAH1 protospacer, position 2 of the PAH2 protospacer) has the potential for bystander editing. The MIT specificity score of PAH1 in its wild-type version is 98 (out of 100), and the score of PAH2 in its wild-type version is 95.

FIG. 3A-3B. Prime editing to introduce PAH c.842C>T variant into HuH-7 human hepatoma cells, followed by adenine base editing to correct the variant. (FIG. 3A) Generation of a homozygous PAH P281L HuH-7 cell line with prime editing. Top=sequence from wild-type HuH-7 cells. Middle=sequence from a pool of HuH-7 cells into which prime editor was introduced by transient transfection of plasmids. Bottom left=sequence from a clonal HuH-7 cell line demonstrating homozygosity for the P281L variant (HuH-7 P281L cells). (FIG. 3B) A-to-G editing following transient transfection of HuH-7 P281L cells with plasmids encoding varied ABE/gRNA sets. ABE8.8 in combination with a validated PCSK9 gRNA was used as a reference.

FIGS. 4A-4F. Dual-AAV-mediated vs. single-AAV-mediated adenine base editing in the livers of wild-type and PCSK9-humanized mice in vivo. (FIG. 4A) Dose-dependent editing of Pcsk9 is similar with SpABE8e (split-intein, two AAVs) and SaKKH ABE8e (one AAV). (FIG. 4B) Editing of PCSK9 with SaKKH ABE8e in humanized mice. (FIGS. 4C and 4D) Reduction of blood PCSK9 protein levels with all ABE treatments. (FIGS. 4E and 4F) Reduction of blood cholesterol levels with all ABE treatments.

FIGS. 5A-5D. LNP-mediated or eVLP-mediated adenine base editing in the livers of mice or non-human primates (NHPs) in vivo. (FIG. 5A) Dose-dependent editing of mouse Pcsk9 with LNPs with ABE8.8 mRNA+Pcsk9 gRNA. (FIG. 5B) Editing of NHP PCSK9 two weeks after treatment with LNPs with ABE8.8 mRNA+PCSK9 gRNA. (FIG. 5C) Reduction of blood PCSK9 protein levels in NHPs (control n=2, treated n=4). (FIG. 5D) Editing of mouse Pcsk9 one week after treatment with eVLPs with ABE8e protein+Pcsk9 gRNA.

FIG. 6. Scheme to generate exon-humanized PKU mice with the PAH c.842C>T (P281L) variant. “T rs5030851”=PAH c.842C>T variant.

FIGS. 7A-7B. Generation of minimally humanized PKU mice with the PAH c.842C>T (P281L) variant. (FIG. 7A) Sanger sequencing chromatograms showing the generation of a humanized mouse model via Cas9-mediated homology-directed repair in mouse zygotes. At the top is sequence from a wild-type C57BL/6J mouse. At the bottom is sequence from a mouse homozygous for the humanized Pah P281L allele. The red arrow indicates the site of the P281L variant, and the black arrows indicate the sites of synonymous changes that humanize the local region of the mouse Pah gene. (FIG. 7B) Age-matched colony mates that are homozygous or heterozygous for the humanized P281L allele. The left picture shows two homozygous mice with PKU as evidenced by hypopigmentation of the fur and two control heterozygous mice with normal fur color, immediately prior to treatment. The right picture shows the two homozygous mice and two heterozygous mice 8 weeks after the homozygous mice received LNP treatment, with normalization of fur color.

FIG. 8. A-to-G editing observed in dose-response studies with HuH-7 P281L cells treated with LNPs formulated with ABE8.8 mRNA and either PAH1 gRNA (left) or PAH2 gRNA (right). On-target editing includes all outcomes in which corrective editing of the P281L variant was achieved, irrespective of bystander editing (n=3 biological replicates; mean±standard deviation for each dose).

FIGS. 9A-9D. Assessment of off-target editing in primary human hepatocytes, primary cynomolgus (cyno) monkey hepatocytes, and cynomolgus monkey (NHP) liver in vivo. (FIG. 9A) (SEQ ID NO: 11) Candidate sites for guide RNA-dependent DNA editing nominated by ONE-seq and Digenome-seq. The box in the right panel highlights the C5 off-target site; the asterisks in the middle panel indicate human ABE-Digenome-seq-nominated sites that overlap with human ONE-seq in the left panel. (FIG. 9B) Editing at the on-target PCSK9 site and the candidate off-target PCSK9 sites in primary human hepatocytes from four individual donors (n=4 LNP-treated and 4 untreated samples for each site). (FIG. 9C; top panel; n=3 treated and 3 untreated biological replicates) Editing at the on-target PCSK9 site and the candidate off-target PCSK9 sites in primary cyno hepatocytes and in NHP liver (FIG. 9D; bottom panel; n=3 LNP-treated animals and 3 animals that received phosphate-buffered saline).

FIGS. 10A-10C. On-target or off-target editing at top candidate sites nominated by ONE-seq calculated as net A-to-G editing (proportion of sequencing reads with alteration of one or more A bases to G in treated samples versus untreated samples) in HuH-7 P281L cells that underwent transient transfection of plasmids (top two graphs; n=2 treated and 2 untreated biological replicates; mean±standard deviation for each site) or lentiviral infection with a Lenti-seq library (bottom graph). Sites for which targeted amplicon sequencing was unsuccessful are not shown. (FIG. 10A) Targeted amplicon sequencing results with ABE8.8/PAH1. (FIG. 10B) Targeted amplicon sequencing results with ABE8.8/PAH2. (FIG. 10C) Lenti-seq results with ABE8.8/PAH2.

FIGS. 11A-11E. Adenine base editing for correction of the PAH P281L variant in humanized PKU mice in vivo. (FIG. 11A) Short-term changes in the blood phenylalanine level in homozygous PKU mice following treatment with 2.5 mg/kg dose of ABE8.8/PAH1 LNPs, comparing levels at various time points up to 7 days following treatment to levels in untreated control PKU and non-PKU heterozygous age-matched (8 weeks of age) colony mates (n=1 sample per animal at each time point). (FIG. 11B) Short-term changes in the blood phenylalanine level in compound heterozygous PKU mice following treatment with 2.5 mg/kg dose of ABE8.8/PAH1 LNPs, comparing with control heterozygous non-PKU age-matched (4 weeks of age) colony mates (n=1 sample per animal at each time point). (FIG. 11C) Short-term changes in the blood phenylalanine level in homozygous and compound heterozygous PKU mice (10 weeks of age) following treatment with 2.5 mg/kg dose of ABE8.8/PAH2 LNPs (n=1 sample per animal at each time point). (FIG. 11D), Long-term changes in the blood phenylalanine level in homozygous PKU mice following treatment with 2.5 mg/kg dose of ABE8.8/PAH1 LNPs, comparing levels at various time points up to 10 weeks following treatment to levels in control heterozygous non-PKU age-matched (8 weeks of age) colony mates (n=1 sample per animal at each time point). (FIG. 11E) A-to-G editing in various mouse organs, assessed 1 to 2 weeks following treatment with 2.5 mg/kg dose of LNPs (on the left, mean±standard deviation for each organ; on the right, whole-liver editing for each individual mouse). For compound heterozygous mice, each displayed number is % edited P281L alleles (editable alleles) divided by two.

FIGS. 12A-12B. Assessment of mouse liver following LNP treatment. (FIG. 12A) Long-term changes in the blood aspartate aminotransferase (AST) level (top) and alanine aminotransferase (ALT) level (bottom) in homozygous PKU mice following treatment with 2.5 mg/kg dose of ABE8.8/PAH1 LNPs, comparing levels at various time points up to 8 weeks following treatment to levels in untreated heterozygous non-PKU age-matched (8 weeks of age) colony mates (n=1 sample per animal at each time point). (FIG. 12B) Liver histology (hematoxylin/eosin staining) at 20× magnification upon necropsy at 1 week after LNP treatment of humanized PKU mouse (bottom) compared to age-matched, untreated non-PKU mouse (top). Lines indicate distance of 50 μm.

### DETAILED DESCRIPTION OF THE INVENTION

In vivo gene editing is an emerging therapeutic approach to making DNA modifications in the body of a patient, such as in the liver. Gene-editing methods include CRISPR-Cas9 and -Cas12 nucleases, CRISPR cytosine base editors, CRISPR adenine base editors, and CRISPR prime editors. CRISPR base editors are an attractive gene-editing modality because they function efficiently for introducing precise targeted alterations without the need for double-strand breaks, in contrast to CRISPR-Cas9 and other gene-editing nucleases (e.g., Cas12). Adenine base editors (ABEs) can induce targeted A→G edits in DNA (T→C on the opposing strand). Each ABE uses its core Cas9 nickase protein with a guide RNA (gRNA) to engage a double-strand protospacer DNA sequence, flanked by a protospacer-adjacent motif (PAM) sequence on its 3′ end. Because ABEs do not make double-strand breaks, they have only minimal risk of inducing large deletions, chromosomal abnormalities, and chromothripsis (shattering); instead, each ABE uses an evolved deoxyadenosine deaminase domain—typically fused to the N-terminal end of the Cas9 nickase—to chemically modify an adenosine nucleoside on one DNA strand, which (in combination with nicking of the other strand) enables highly precise and efficient A→G transition mutations at the targeted site.

The activity window of each ABE typically ranges across several positions within the protospacer DNA sequence (e.g., the ABE8.8 window ranges from position 3 to position 9, with peak editing observed at position 6 of the protospacer), with different ABEs having different windows. ABEs have the potential to edit any adenine within the window, which could include a desired target adenine but also undesired additional adenines (bystander edits). Published ABEs with Streptococcus pyogenes Cas9 nickase include so-called eighth-generation ABEs (harboring optimized deaminase domains resulting from eight rounds of molecular evolution) the most commonly used to date are ABE8.8, ABE8.20, and ABE8e—and circularly permuted or inlaid ABEs, in which the deaminase domain is embedded within a loop of the Cas9 nickase protein, rather than fused to the N-terminal end, which has the effect of shifting the editing window further towards the 3′ end of the protospacer sequence. Similar ABEs with Cas9 nickase from other bacterial species (e.g., Staphylococcus aureus) have been reported. As a general rule, ABEs display highly variable levels of activity across different genomic loci in different cell types, and empirical testing is mandatory to determine whether a given ABE with a given gRNA will edit efficiently at a given target site in a given cell type.

The present invention provides compositions and methods for adenine base editing to permanently correct one of the top 5 most common pathogenic variants, the PAH c.842C>T (P281L) variant, in human hepatocytes. The PAH c.842C>T variant has its highest prevalence in populations in the Middle East, Russia, and Europe but is widespread across the globe. Patients homozygous for this variant do not respond at all to sapropterin, limiting their treatment options and making a curative in vivo base editing therapy particularly compelling.

### Definitions

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

A “monogenic disease” or a “monogenic disorder” is a condition determined by the interaction of a single pair of genes. This is in contrast to a polygenic condition wherein several genes are involved. In humans, monogenic diseases occur less frequently than the polygenic disease. It is also less complicated than the latter and may follow a pattern based on Mendelian inheritance. Monogenic disorders can adversely impact a number of biological systems.

Phenylketonuria (PKU) is a classic “monogenic” autosomal recessive disease in which mutation at the human phenylalanine hydroxylase (PAH) locus impairs the function of the enzyme phenylalanine hydroxylase (enzymic phenotype), thereby causing the attendant hyperphenylalaninemia (metabolic phenotype) and the resultant intellectual disability (cognitive phenotype). Other symptoms include seizures, tremors, hyperactivity, stunted growth, or shaking and trembling, skin conditions including eczema, as well as musty odor of the urine, breath, or skin. 0.45 million individuals have PKU, with global prevalence 1:23,930 live births (range 1:4,500 [Italy]-1:125,000 [Japan]). More than 1280 variants in the phenylalanine hydroxylase PAH gene are responsible for a broad spectrum of phenylketonuria (PKU) phenotypes. While genotype-phenotype correlation is ˜88%, additional factors play a role. These include tetrahydrobiopterin (BH₄), the PAH co-chaperone DNAJC12, phosphorylation of the PAH residues, and epigenetic factors. There is presently no cure for PKU, with the exception of liver transplantation. Here the direct correction of the causative mutation PAH c.842C>T via base editing, also known as p.Pro281Leu, in liver cells is described. This is the fifth most common PKU associated gene mutation. Subjects harboring this mutation do not respond to BH4 supplementation therapy.

The term “deaminase” or “deaminase domain” refers to a protein or enzyme that catalyzes a deamination reaction. In some embodiments, the deaminase is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium.

In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

It should be appreciated, however, that additional adenosine deaminases useful in the present application would be apparent to the skilled artisan and are within the scope of this disclosure.

The term “base editor (BE),” or “nucleobase editor (NBE)” refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating an adenine (A) in DNA. In some embodiments, the base editor is a fusion protein comprising a nucleic acid programmable DNA binding protein (napDNAbp) fused to an adenosine deaminase. In some embodiments, the base editor is a Cas9 protein fused to an adenosine deaminase. In some embodiments, the base editor is a Cas9 nickase (nCas9) fused to an adenosine deaminase. In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain.

“Prime editing” directly introduces new genetic information into a targeted DNA site. Typically editing is effected by a fusion protein, consisting of a catalytically impaired Cas9 endonuclease fused to an engineered reverse transcriptase enzyme, and a prime editing guide RNA (pegRNA), capable of identifying the target site and providing the new genetic information to replace the target DNA nucleotides. Using this technique targeted insertions, deletions, and base-to-base conversions without the need for double strand breaks (DSBs) or donor DNA templates can be introduced into the targeted nucleic acid molecule.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an adenosine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

As used herein the term “wild-type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild-type or a comprises non naturally occurring components.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4.sup.th ed., Cold Spring Harbor Laboratory Press, Cold Spring Harbor, N.Y. (2012)).

The term “uracil glycosylase inhibitor” or “UGI,” as used herein, refers to a protein that is capable of inhibiting a uracil-DNA glycosylase base-excision repair enzyme. In some embodiments, the UGI proteins provided herein include fragments of UGI and proteins homologous to a UGI or a UGI fragment. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence encoding UGI.

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for their disclosure of exemplary nuclear localization sequences.

The term “nucleic acid programmable DNA binding protein” or “napDNAbp” refers to a protein that associates with a nucleic acid (e.g., DNA or RNA), such as a guide nucleic acid, that guides the napDNAbp to a specific nucleic acid sequence. For example, a Cas9 protein can associate with a guide RNA that guides the Cas9 protein to a specific DNA sequence that has complementarity to the guide RNA. In some embodiments, the napDNAbp is a class 2 microbial CRISPR-Cas effector. In some embodiments, the napDNAbp is a Cas9 domain, for example a nuclease active Cas9, a Cas9 nickase (nCas9), or a nuclease inactive Cas9 (dCas9). Examples of nucleic acid programmable DNA binding proteins include, without limitation, Cas9 (e.g., dCas9 and nCas9), CasX, CasY, Cpf1, C2c1, C2c2, C2C3, and Argonaute. It should be appreciated, however, that nucleic acid programmable DNA binding proteins also include nucleic acid programmable proteins that bind RNA. For example, the napDNAbp may be associated with a nucleic acid that guides the napDNAbp to an RNA. Other nucleic acid programmable DNA binding proteins are also within the scope of this disclosure, though they may not be specifically listed in this disclosure.

The term “Cas9” or “Cas9 domain” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). CRISPR (clustered regularly interspaced short palindromic repeat) is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference).

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1) or to a Cas9 from any other organism.

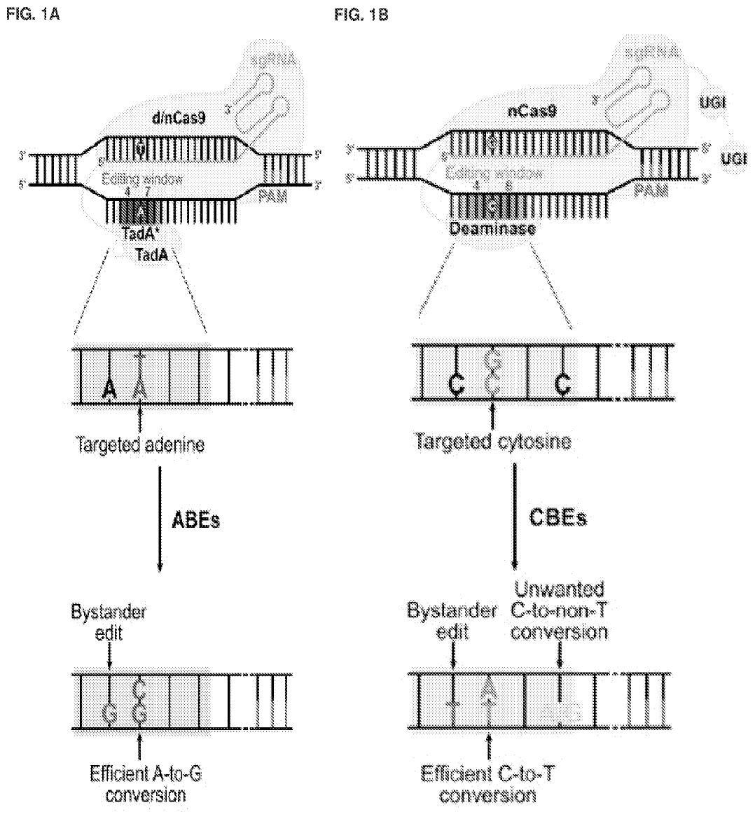

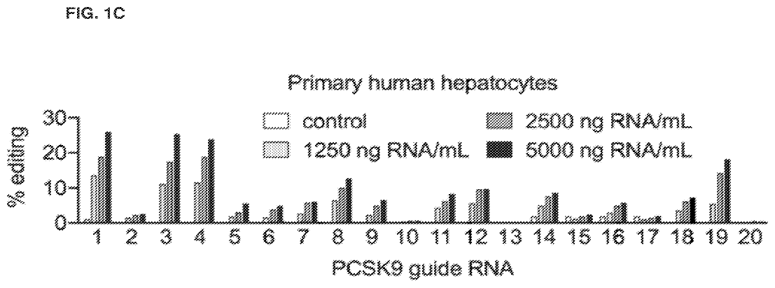

In some embodiments, dCas9 corresponds to, or comprises in part or in whole, a Cas9 amino acid sequence having one or more mutations that inactivate the Cas9 nuclease activity. Such Cas9 variants are able to generate a single-strand DNA break (nick) at a specific location based on the gRNA-defined target sequence, leading to repair of the non-edited strand, ultimately resulting in a T to C change on the non-edited strand. A schematic representation of this process is shown in FIG. 1B. Briefly, and without wishing to be bound by any particular theory, the A of a A⋅T base pair can be deaminated to a inosine (I) by an adenosine deaminase, e.g., an engineered adenosine deaminase that deaminates an adenosine in DNA. Nicking the non-edited strand, having the T, facilitates removal of the T via mismatch repair mechanisms. A UGI domain or a catalytically inactive inosine-specific nuclease (dISN) may inhibit inosine-specific nucleases (e.g., sterically) thereby preventing removal of the inosine (I).

In some embodiments, the nucleic acid programmable DNA binding protein (napDNAbp) of any of the fusion proteins provided herein may be a CasX or CasY protein. In some embodiments, the napDNAbp is a CasX protein. In some embodiments, the napDNAbp is a CasY protein.

The term “effective amount,” as used herein, refers to an amount of a biologically active agent that is sufficient to elicit a desired biological response. For example, in some embodiments, an effective amount of a nucleobase editor may refer to the amount of the nucleobase editor that is sufficient to induce mutation of a target site specifically bound and mutated by the nucleobase editor. In some embodiments, an effective amount of a fusion protein provided herein, e.g., of a fusion protein comprising a nucleic acid programmable DNA binding protein and a deaminase domain (e.g., an adenosine deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nucleobase editor, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4.sup.th ed., Cold Spring Harbor Laboratory Press, Cold Spring Harbor, N.Y. (2012)), the entire contents of which are incorporated herein by reference.

The term “RNA-programmable nuclease,” and “RNA-guided nuclease” are used interchangeably herein and refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), the entire contents of which is incorporated herein by reference. Other examples of gRNAs (e.g., those including domain 2) can be found in U.S. Provisional Patent Application Ser. No. 61/874,682, filed Sep. 6, 2013, entitled “Switchable Cas9 Nucleases And Uses Thereof,” and U.S. Provisional Patent Application Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al., Multiplex genome engineering using CRISPR/Cas systems. Science 339, 819-823 (2013); Mali, P. et al., RNA-guided human genome engineering via Cas9. Science 339, 823-826 (2013); Hwang, W. Y. et al., Efficient genome editing in zebrafish using a CRISPR-Cas system. Nature Biotechnology 31, 227-229 (2013); Jinek, M. et al., RNA-programmed genome editing in human cells. eLife 2, e00471 (2013); Dicarlo, J. E. et al., Genome engineering in Saccharomyces cerevisiae using CRISPR-Cas systems. Nucleic Acids Research (2013); Jiang, W. et al. RNA-guided editing of bacterial genomes using CRISPR-Cas systems. Nature Biotechnology 31, 233-239 (2013); the entire contents of each of which are incorporated herein by reference).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a sequence within a nucleic acid molecule that is deaminated by a deaminase or a fusion protein comprising a deaminase, (e.g., a dCas9-adenosine deaminase fusion protein provided herein).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

In certain embodiments, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a gRNA is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism.

Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994). Methods of non-viral delivery of nucleic acids include lipofection, nucleofection, microinjection, biolistics, virosomes, liposomes, immunoliposomes, polycation or lipid-nucleic acid conjugates, lipid nanoparticles, artificial virions, virus-like particles, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g., in vitro or ex vivo administration) or target tissues (e.g., in vivo administration).

Methods to deliver gene editing agents in vivo as ribonucleoproteins is another approach and provides safety advantages over nucleic acid delivery approaches. Engineered DNA-free virus-like particles (eVLPs) have been developed that efficiently package and deliver base editor or Cas9 ribonucleoproteins. By engineering VLPs to overcome cargo packaging, release, and localization bottlenecks, fourth-generation eVLPs have been developed that mediate efficient base editing in several primary mouse and human cell types. Using different glycoproteins in eVLPs alters their cellular tropism. Single injections of eVLPs into mice support therapeutic levels of base editing in multiple tissues, reducing serum Pcsk9 levels 78% following 63% liver editing, and partially restoring visual function in a mouse model of genetic blindness. In vitro and in vivo off-target editing from eVLPs was virtually undetected, an improvement over AAV or plasmid delivery. Thus, eVLPs provide promising vehicles for therapeutic macromolecule delivery that combine key advantages of both viral and nonviral delivery. See S. Banskota et al. Cell 185: 250-265 (2021).

The preparation of lipid nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). Other lipid nanoparticle formulations are disclosed in U.S. Pat. Nos. 11,066,355; 11,059,807; US patent publications 2021/0106538 and 2021/0113466.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue.

Retroviral vectors comprise cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Several different AAV serotypes have been used to advantage for transduction of mammalian cells, these include, for example AAV1, AAV2, AAV4, AAV5, AAV6, AAV7, AAV8, and AAV9 that have different tropisms for cell types of interest. Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). In certain preferred embodiments, the viral vector is a split AA8 vector or a split AAV9 vector.

Packaging cells are typically used to form virus particles that are capable of infecting a host cell. Such cells include HEK 293 cells, which package adenovirus, and y2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line.

For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

In one aspect, the invention provides for methods of modifying a target polynucleotide in a eukaryotic cell. In some embodiments, the method comprises allowing an adenine base editor (ABE) CRISPR complex to bind to the target polynucleotide to effect correction of a mutation in said target polynucleotide thereby modifying the target polynucleotide, wherein the CRISPR complex comprises the ABE CRISPR enzyme complexed with a gRNA hybridized to a target sequence within said target polynucleotide.

In one aspect, the invention provides kits containing any one or more of the elements disclosed in the above methods and compositions. In some embodiments, the kit comprises a vector system or components for an alternative delivery system such as those described above and instructions for using the kit. In some embodiments, the vector or delivery system comprises an ABE CRISPR enzyme complexed with a gRNA for base editing of a target nucleic acid.

The kit can contain a lipid nanoparticle formulation encapsulating the appropriate base editor and at least one gRNA. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a gRNA sequence for insertion into a vector so as to operably link the gRNA sequence and a regulatory element. In some embodiments, the kit comprises a homologous recombination template polynucleotide.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types in methods of gene therapy.

As used herein, the term “metabolic gene” is defined as an inherited single gene anomaly, i.e., a single gene coding for an enzyme is defective, and that defect causes an enzyme deficiency. The enzyme deficiency produces an inherited metabolic disease or disorder, of which a subtype is an inborn error of metabolism. Most single gene anomalies are autosomal recessive, i.e., two defective copies of the gene must be present for the disease or trait to develop. Non-limiting examples of metabolic disorders include glucose metabolism disorders, lipid metabolism disorders, malabsorption syndromes, metabolic brain diseases, calcium metabolism disorders, DNA repair-deficiency disorders, hyperlactemia, iron metabolism disorders, metabolic syndrome X, inborn error of metabolism, phosphorus metabolism disorders, and acid-base imbalance. Inherited metabolic diseases previously were classified as disorders of carbohydrate metabolism, amino acid metabolism, organic acid metabolism, or lysosomal storage diseases; however new inherited disorders of metabolism have been discovered and the categories have multiplied. Certain major classes of congenital metabolic diseases include disorders of carbohydrate metabolism, e.g., glycogen storage disease, glucose-6-phosphate dehydrogenase (G6PD) deficiency (resulting from a mutation in the G6PD gene); disorders of amino acid metabolism, e.g., phenylketonuria, maple syrup urine disease, glutaric acidemia type 1; urea cycle disorder (urea cycle defects), e.g., carbamoyl phosphate synthetase I deficiency; disorders of organic acid metabolism (organic acidurias), e.g., alcaptonuria, 2-hydroxyglutaric acidurias; disorders of fatty acid oxidation and mitochondrial metabolism; e.g., medium-chain acyl-coenzyme A dehydrogenase deficiency (often called “MCADD”) (caused by mutations in the ACADM gene, which results in medium-chain fatty acids not being metabolized properly and leads to lethargy and hypoglycemia); disorders of porphyrin metabolism, e.g., acute intermittent porphyria; disorders of purine or pyrimidine metabolism, e.g., Lesch-Nyhan syndrome (caused by mutations in the hypoxanthine phosphoribosyltransferase 1 [HPRT1] gene and inherited in an X-linked recessive manner); disorders of steroid metabolism, e.g., lipoid congenital adrenal hyperplasia, congenital adrenal hyperplasia; disorders of mitochondrial function, e.g., Keams-Sayre syndrome; disorders of peroxisomal function, e.g., Zellweger syndrome (caused by mutations in genes encoding peroxins, e.g., PEX1, PEX2, PEX3, PEX5, PEX6, PEX10, PEX12, PEX13, PEX14, PEX16, PEX19, or PEX26 genes); and lysosomal storage disorders, e.g., Gaucher's disease (of which there are three subtypes, all of which are autosomal recessive) and Niemann-Pick disease (has an autosomal recessive inheritance pattern; Niemann-Pick types A and B are caused by a mutation in the Sphingomyelin phosphodiesterase 1 [SMPD1] gene; mutations in NPC1 gene or NPC2 gene cause Niemann-Pick disease, type C [NPC], which affects a protein used to transport lipids; Niemann-Pick type D shares a specific mutation in the NPC1 gene, patients having type D share a common Nova Scotian ancestry).

In certain aspects, an adenine base editor (ABE) complex for programming conversion of adenine to guanine in a patient in need thereof is provided where the patient has a target DNA molecule harboring a mutation associated with phenylketonuria. An exemplary ABE complex includes a modified TadA enzyme, a catalytically impaired Cas9 protein and at least one single guide RNA (sgRNA) which directs said ABE complex to said mutated target DNA molecule, which upon contact converts adenosine in said mutation to inosine, thereby catalyzing an A⋅T to G⋅C transition following DNA repair or DNA replication.

The activity window of each ABE typically ranges across several positions within the protospacer DNA sequence (e.g., the ABE8.8 window ranges from position 3 to position 9, with peak editing observed at position 6 of the protospacer), with different ABEs having different windows (Anzalone et al., 2020). ABEs have the potential to edit any adenine within the window, which could include a desired target adenine but also undesired additional adenines (bystander edits). Published ABEs with Streptococcus pyogenes Cas9 nickase include so-called eighth-generation ABEs (harboring optimized deaminase domains resulting from eight rounds of molecular evolution)—the most commonly used to date are ABE8.8, ABE8.20, and ABE8e—and circularly permuted or inlaid ABEs, in which the deaminase domain is embedded within a loop of the Cas9 nickase protein, rather than fused to the N-terminal end, which has the effect of shifting the editing window further towards the 3′ end of the protospacer sequence (Gaudelli et al., 2020; Richter et al., 2020; Chu et al., 2021). Similar ABEs with Cas9 nickase from other bacterial species (e.g., Staphylococcus aureus) have been reported (Gaudelli et al., 2020; Richter et al., 2020).

The following materials and methods are provided to facilitate the practice of the present invention.

To generate lentiviral constructs, the Cas9 gene in the lentiCRISPR v2 plasmid (Addgene #52961) was replaced with the ABE8.20 gene from the ABE8.20-m plasmid (Addgene #136300) using standard molecular biology techniques. The gRNA sequence along with an 18-nucleotide barcode and the 73-nucleotide exogenous target sequence (either the PAH genomic sequence bearing the c.842C>T variant, or the PCSK9 genomic sequence spanning the exon 1 splice donor) was generated by DNA synthesis (GENEWIZ) and inserted via Gibson cloning using the BsmBI restriction sites already present within the U6-gRNA cassette in the plasmid (https://media.addgene.org/data/plasmids/52/52961/52961-attachment_B3xTwlaObkYD.pdf).

For prime editing, the pCMV-PEmax-P2A-hMLH1dn plasmid (Addgene #174828) was used to express the prime editor (PE), the pU6-tevopreq1-GG-acceptor plasmid (Addgene #174038) was used to express the prime editing guide RNA (pegRNA)—for insertion of the PAH c.842C>T variant—following Gibson cloning of the oligonucleotide-synthesized pegRNA sequence, and the pGuide plasmid (Addgene #64711) was used to express the nicking guide RNA (ngRNA) following subcloning of the oligonucleotide-synthesized ngRNA sequence. For base editing, a variety of adenine base editor (ABE)-expressing plasmids were used: ABE8.8-m (Addgene #136294), ABE8.13-m (Addgene #136296), ABE8.17-m (Addgene #136298), ABE8.20-m (Addgene #136300), ABE8e (Addgene #138489), CP1028-ABE8e (Addgene #138492), or CP1041-ABE8e (Addgene #138493). The pGuide plasmid (Addgene #64711) was used to express each accompanying guide RNA (specific for the PAH c.842C>T variant or the PCSK9 exon 1 splice donor) following subcloning of the oligonucleotide-synthesized gRNA sequence.

HuH-7 human hepatoma cells were obtained from the Japanese Collection of Research Bioresources (JCRB) Cell Bank and maintained in culture with DMEM containing 1 g/L glucose and supplemented with 10% FBS (Thermo Fisher). On the day prior to transfection, the cells were split and replated into 6-well dishes at 3.5×10⁵ cells/well to achieve ≈80% confluence at the time of transfection. For prime editing, each well was transfected with 9 μL TransIT-LT1 Transfection Reagent mixed with 1.5 μg of the PE-expressing plasmid, 0.75 μg of the pegRNA-expressing plasmid, and 0.75 μg of the ngRNA-expressing plasmid, using the protocol described above. Three days following transfection, the cells were split and replated dilutely in 10-cm plates as single cells in order to foster the growth of distinct colonies. After 1 week of growth, individual colonies were picked, transferred to a 96-well plate, and subsequently split and expanded to plates with larger wells, with some of the split cells diverted for harvesting of genomic DNA with the DNeasy Blood & Tissue Kit. A clonal cell line confirmed by Sanger sequencing and next-generation sequencing to be homozygous for the PAH c.842C>T variant was further expanded and used for subsequent base editing experiments.

For base editing experiments, each well of a 6-well plate was transfected with 9 μL TransIT-LT1 Transfection Reagent mixed with 2 μg of the ABE-expressing plasmid and 1 μg of the gRNA-expressing plasmid. The cells were removed from the plates by scraping 3 days after transfection, washed with phosphate-buffered saline, and harvested for genomic DNA with the DNeasy Blood & Tissue Kit.

PCR amplification of the target sequences (endogenous PAH locus or endogenous PCSK9 locus) in genomic DNA samples from lentivirus-treated primary human hepatocytes or transfected HuH-7 cells was performed using NEBnext High-Fidelity 2×PCR Master Mix (New England Biolabs) with locus-specific primers containing 5′ Nextera adaptor sequences (Illumina), followed by purification of the PCR amplicons with the Sequalprep Normalization Plate kit (Thermo Fisher) or NGS Normalization 96-Well Kit (Norgen Biotek). A second round of PCR with the Nextera XT Index Kit V2 Set A and/or Nextera XT Index Kit V2 Set D (Illumina), followed by purification with the Sequalprep Normalization Plate Kit or NGS Normalization 96-Well Kit, generated barcoded libraries, which were pooled and quantified using a Qubit 3.0 Fluorometer. After denaturation, dilution to 10 pM, and supplementation with 15% PhiX, the pooled libraries underwent paired-end next-generation sequencing on an Illumina MiSeq System. The amplicon sequencing data were analyzed with CRISPResso2 (https://crispresso.pinellolab.partners.org/). In some cases, PCR amplicons were subjected to confirmatory Sanger sequencing, performed by GENEWIZ, with editing frequencies estimated from the chromatograms. MIT specificity scores for gRNAs were determined using CRISPOR. See crispor.tefor.net/.

100-mer PAH1 and PAH2 gRNAs were chemically synthesized under solid phase synthesis conditions by a commercial supplier (Agilent) with end-modifications as well as heavy 2′-O-methylribosugar modification:

PAH1,
(SEQ ID NO: 101)
5′-mU*mC*mA*CAGUUCGGGGGUAUACAGUUUUAGAmGmCmUmAmGm
AmAmAmUmAmGmCAAGUUAAAAUAAGGCUAGUCCGUUAUCAmAmCmUm
UmGmAmAmAmAmAmGmUmGmGmCmAmCmCmGmAmGmUmCmGmGmUmGm
CmU*mU*mU*mU-3′;
PAH2,
(SEQ ID NO: 102)
5′-mC*mA*mC*AGUUCGGGGGUAUACAUGUUUUAGAmGmCmUmAmGm
AmAmAmUmAmGmCAAGUUAAAAUAAGGCUAGUCCGUUAUCAmAmCmUm
UmGmAmAmAmAmAmGmUmGmGmCmAmCmCmGmAmGmUmCmGmGmUmGm
CmU*mU*mU*mU-3′;

where “m” and * respectively indicate 2′-O-methylation and phosphorothioate linkage. ABE8.8 mRNA was produced via in vitro transcription (IVT) and purification. In brief, a plasmid DNA template containing a codon-optimized ABE8.8 coding sequence and a 3′ polyadenylate sequence was linearized. An IVT reaction containing linearized DNA template, T7 RNA polymerase, NTPs, and cap analog was performed to produce mRNA containing N1-methylpseudouridine. After digestion of the DNA template with DNase I, the mRNA product underwent purification and buffer exchange, and the purity of the final mRNA product was assessed with spectrophotometry and capillary gel electrophoresis. Elimination of double-stranded RNA contaminants was assessed using dot blots and transfection into human dendritic cells. Endotoxin content was measured using a chromogenic Limulus amebocyte lysate (LAL) assay; all assays were negative.

Lipid nanoparticles (LNPs) were formulated as previously described, with the lipid components (SM-102, 1,2-distearoyl-sn-glycero-3-phosphocholine, cholesterol, and a PEG-lipid) being rapidly mixed with an aqueous buffer solution containing ABE8.8 mRNA and either PAH1 gRNA or PAH2 gRNA in a 1:1 ratio by weight in 25 mM sodium acetate (pH 4.0). The resulting LNP formulations were subsequently dialyzed against sucrose-containing buffer, concentrated using Amicon Ultra-15 mL Centrifugal Filter Units (Millipore Sigma), sterile-filtered using 0.2-μm filters, and frozen until use. The LNPs had particle sizes of 69-89 nm (Z-Ave, hydrodynamic diameter), with a polydispersity index of <0.21 as determined by dynamic light scattering (Malvern NanoZS Zetasizer) and 90%-100% total RNA encapsulation as measured by the Quant-iT Ribogreen Assay (Thermo Fisher Scientific).

HuH-7 cells were maintained in Dulbecco's modified Eagle's medium (containing 4 mM L-glutamine and 1 g/L glucose) with 10% fetal bovine serum and 1% penicillin/streptomycin at 37° C. with 5% CO₂. HuH-7 cells were seeded on 6-well plates (Corning) at 3.5×10⁵ cells per well. At 16-24 hours after seeding, cells were transfected at approximately 80-90% confluency with 9 μL TransIT®-LT1 Transfection Reagent (MIR2300, Mirus), 2 μg base editor plasmid, and 1 μg gRNA plasmid per well according to the manufacturer's instructions. In other experiments, LNPs were added at various doses (quantified by the total amount of RNA within the LNPs) directly to the media. In other experiments, RNA transfection was performed: for high dose, 3 μL Lipofectamine MessengerMAX (Thermo Fisher Scientific), 0.5 μg ABE8.8 mRNA, and 0.5 μg gRNA per well was transfected according to the manufacturer's instructions; for medium dose, 1 μL MessengerMAX, 0.17 μg ABE8.8 mRNA, and 0.17 μg gRNA per well was transfected; for low dose, 0.36 μL MessengerMAX, 0.06 μg ABE8.8 mRNA, and 0.06 μg gRNA per well was transfected. Cells were cultured for 72 hours after transfection, and then media were removed, cells were washed with 1×DPBS (Corning), and genomic DNA was isolated using the DNeasy Blood and Tissue Kit (QIAGEN) according to the manufacturer's instructions.

HuH-7 cells in a well of a 6-well plate were transfected with 9 μL TransIT®-LT1 Transfection Reagent, 1.5 μg PEmax plasmid, 0.75 μg epegRNA-expressing plasmid, and 0.75 μg nicking gRNA plasmid. Cells were dissociated with trypsin 48 hours post-transfection and replated onto 10-cm plates (5,000 cells/plate) with conditioned medium to facilitate recovery, and genomic DNA was isolated from the remainder of the cells as a pool to perform PCR and Sanger sequencing of the PAH P281L site. Single cells were permitted to expand for 7-14 days to establish clonal populations. Colonies were manually picked and replated into individual wells of a 96-well plate. Genomic DNA was isolated from individual clones, and PCR and Sanger sequencing was performed to identify homozygous P281L HuH-7 clones. One representative clone was expanded for use in subsequent studies.

A PKU mouse model with one or more humanized Pah P281L alleles was generated using in vitro transcribed Cas9 mRNA, a synthetic gRNA (spacer sequence 5′-UAGCUGAAGAAUGAUACUUA-3′ (SEQ ID NO: 14)) (Integrated DNA Technologies), and a synthetic single-strand DNA oligonucleotide (Integrated DNA Technologies) with homology arms matching the target site and harboring the P281L variant and synonymous variants (bold with underline): 5′TGCTGGCTTACTGTCGTCTCGAGATTTCTTGGGTGGCCTGGCCTTCCGAGTCTTCCA CTGCACACAGTACATTAGGCATGGATCTAAGCCCATGTATACCCCCGAACTGTGAG ATCATTCTTCAGCTACCCCTGCCAACCACAATGGATGCTCAAAGAATGCTGATCAGG CTCATTGCAGGCTGGTCCCCATGATCCAC-3′(SEQ ID NO: 15). The mixture of the 3 components was injected into cytoplasm of fertilized oocytes from C57BL/6J mice at the Penn Vet Transgenic Mouse Core (https://www.vet.upenn.edu/research/core-resources-facilities/transgenic-mouse-core). Genomic DNA samples from founders were screened for knock-in of the desired sequence in the Pah locus via homology-directed repair. Founders with the humanized P281L allele were bred through two generations to obtain homozygous mice. In some founders, indel mutations were present because of non-homologous end-joining within the mouse Pah locus; a non-humanized loss-of-function allele with a 4-bp deletion (ΔGTAA) just distal to the site of the P281L variant was bred together with the humanized P281L allele to generate compound heterozygote mice.

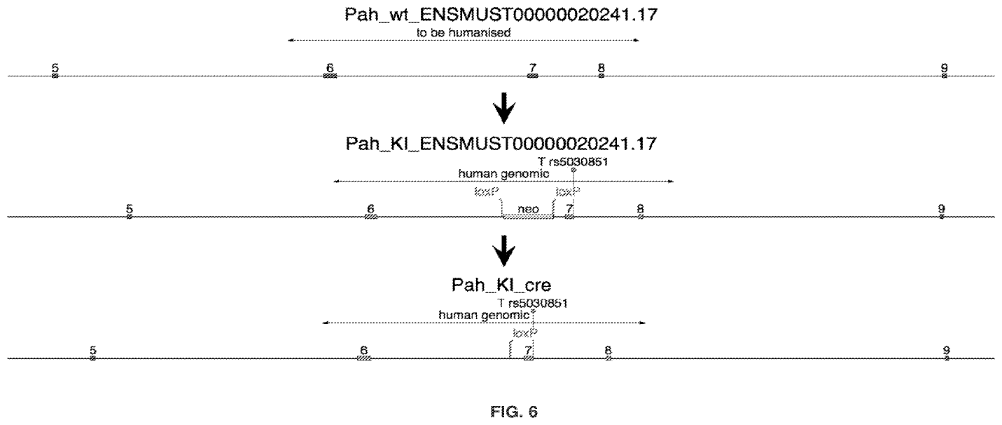

A different PKU mouse model with one or more humanized Pah P281L alleles was generated through the use of homologous recombination in mouse embryonic stem cells, followed by blastocyst injections, generation of chimeras, and subsequent breeding, as schematized in FIG. 6.

Homozygous and compound heterozygous humanized PKU mice, as well as heterozygous humanized non-PKU mice, were generated as littermates/colony-mates via timed breeding, in some cases using wild-type C57BL/6J mice (stock no. 000664) obtained from The Jackson Laboratory. Genotyping was performed using PCR amplification from genomic DNA samples (prepared from clipped tails/ears) followed by next-generation sequencing. Age-matched female and male colony-mates were used for experiments at 4 weeks of age, 8 weeks of age, or 10 weeks of age, with random assignment of animals to various experimental groups when applicable, and with collection and analysis of data performed in a blinded fashion when possible. LNPs were administered to the mice at approximately 2.5 mg/kg doses via retro-orbital injection under anesthesia with 1%-2% inhaled isoflurane. In short-term studies, mice were euthanized at 1 to 2 weeks after treatment, and 8 liver samples (2 from each lobe) and samples of other organs were obtained on necropsy and processed with the DNeasy Blood and Tissue Kit (QIAGEN) as per the manufacturer's instructions to isolate genomic DNA. Next-generation sequencing results from the liver samples were averaged to provide quantification of whole-liver editing. In both short-term and long-term studies, blood samples were collected via the tail tip at various time points (pre-treatment, day 1, day 2, day 3 or 4, day 7, and—when applicable—day 14, day 21, day 28, and every two weeks thereafter), in the early afternoon to account for diurnal variation in blood phenylalanine levels.

The blood phenylalanine levels were measured by an enzymatic method using the Phenylalanine Assay Kit (MAK005, Millipore Sigma) according to the manufacturers' instructions. Briefly, plasma samples were deproteinized with a 10 kDa MWCO spin filter (CLS431478-25EA, Millipore Sigma) and pre-treated with 5 μL of tyrosinase for 10 minutes at room temperature prior to start of the assay. Reaction mixes were made according to the manufacturers' instructions, and the fluorescence intensity of each sample was measured (kex=535/λem=587 nm). Aspartate aminotransferase (AST) (MAK055-1KT, Millipore Sigma) and alanine aminotransferase (ALT) (MAK052-1KT, Millipore Sigma) activities were measured according to the manufacturers' instructions.

Mice were euthanized by CO₂ inhalation at the time of tissue collection. Organs were harvested and fixed in 4% paraformaldehyde. After serial dehydration in ascending concentrations of ethanol and xylene, organs were paraffin-embedded and sectioned, and haematoxylin/eosin staining was performed. For next-generation sequencing (NGS), PCR reactions were performed using NEBNext Polymerase (NEB) using the primer sets 1 with Primer3 v4.1.0 (https://primer3.ut.ee/). The following program was used for all genomic DNA PCRs: 98° C. for 20 seconds, 35×(98° C. for 20 seconds, 57° C. for 30 seconds, 72° C. for 10 seconds), 72° C. for 2 minutes. PCR products were visualized via capillary electrophoresis (QIAxcel, QIAGEN) and then purified and normalized via the Sequalprep Normalization Plate kit (Thermo Fisher) or an NGS Normalization 96-Well Kit (Norgen Biotek Corporation). A secondary barcoding PCR was conducted to add Illumina barcodes (Nextera XT Index Kit V2 Set A and/or Nextera XT Index Kit V2 Set D) using ≈15 ng of first-round PCR product as template, followed by purification and normalization. Final pooled libraries were quantified using a Qubit 3.0 Fluorometer (Thermo Fisher Scientific) and then after denaturation, dilution to 10 pM, and supplementation with 15% PhiX, underwent paired-end sequencing on an Illumina MiSeq System. The amplicon sequencing data were analyzed with CRISPResso2 v2 and custom scripts to quantify editing. For on-target editing, A-to-G editing was quantified at the site of the P281L variant (position 5 of the PAH1 protospacer sequence, position 4 of the PAH2 protospacer sequence) and at the site of the potential bystander adenine (position 3 of the PAH1 protospacer sequence, position 2 of the PAH2 protospacer sequence), with no other adenines present in positions 1 to 10 of either protospacer sequence. For candidate off-target sites, A-to-G editing was quantified throughout the editing window (positions 1 to 10 of the protospacer sequence). In some cases, PCR amplicons were subjected to confirmatory Sanger sequencing, performed by GENEWIZ.

ONE-seq was performed as follows. The human ONE-seq libraries for the PAH1 and PAH2 gRNAs were designed using the GRCh38 Ensembl v98 reference genome (ftp://ftp.ensembl.org/pub/release-98/fasta/homo_sapiens/dna/Homo sapiens.GRCh38.dna.chromosome.{1-22,X,Y,MT}.fa, ftp://ftp.ensembl.org/pub/release-98/fasta/homo_sapiens/dna/Homo_sapiens.GRCh38.dna.nonchromosomal.fa). Sites with up to 6 mismatches and sites with up to 4 mismatches plus up to 2 DNA or RNA bulges, compared to the on-target site, were identified with Cas-Designer v1.2. The final oligonucleotide sequences were generated with a script, and the oligonucleotide libraries were synthesized by Twist Biosciences. Recombinant ABE8.8-m protein was produced by GenScript. Duplicate ONE-seq experiments were performed with each ONE-seq library. Each library was PCR-amplified and subjected to 1.25× AMPure XP bead purification. After incubation at 25° C. for 10 minutes in CutSmart buffer, RNP comprising 769 nM recombinant ABE8.8-m protein and 1.54 μM gRNA was mixed with 100 ng of the purified library and incubated at 37° C. for 8 hours. Proteinase K was added to quench the reaction at 37° C. for 45 minutes, followed by 2× AMPure XP bead purification. The reaction was then serially incubated with EndoV at 37° C. for 30 minutes, Klenow Fragment (New England Biolabs) at 37° C. for 30 minutes, and NEBNext Ultra II End Prep Enzyme Mix (New England Biolabs) at 20° C. for 30 minutes followed by 65° C. for 30 minutes, with 2× AMPure XP bead purification after each incubation. The reaction was ligated with an annealed adaptor oligonucleotide duplex at 20° C. for 1 hour to facilitate PCR amplification of the cleaved library products, followed by 2× AMPure XP bead purification. Size selection of the ligated reaction was performed on a BluePippin system (Sage Science) to isolate DNA of 150-200 bp on a 3% agarose gel cassette, followed by two rounds of PCR amplification to generate a barcoded library, which underwent paired-end sequencing on an Illumina MiSeq System as described above. The analysis pipeline used for processing the data assigned a score quantifying the editing efficiency with respect to the on-target site to each potential off-target site. Sites were ranked based on this ONE-seq score, and the mean ONE-seq score between duplicate experiments was used for site prioritization.

The following examples are provided to illustrate certain embodiments of the invention. They are not intended to limit the invention in any way.

### Example 1

Gene-editing methods and compositions include CRISPR-Cas9 and -Cas12 nucleases (Jinek et al., 2012; Zetsche et al., 2015; Strecker et al., 2019), CRISPR cytosine base editors (Komor et al., 2016), CRISPR adenine base editors (Gaudelli et al., 2017), and CRISPR prime editors (Anzalone et al., 2019). CRISPR base editors are an attractive gene-editing modality because they function efficiently for introducing precise targeted alterations without the need for double-strand breaks, in contrast to CRISPR-Cas9 and other gene-editing nucleases (FIG. 1A, FIG. 1B). Adenine base editors (ABEs) can induce targeted A→G edits in DNA (T→C on the opposing strand). Each ABE uses its core Cas9 nickase protein with a guide RNA (gRNA) to engage a double-strand protospacer DNA sequence, flanked by a protospacer-adjacent motif (PAM) sequence on its 3′ end. Unlike Cas9 and Cas12, ABEs do not make double-strand breaks and have minimal risk of inducing large deletions, chromosomal abnormalities, and chromothripsis (shattering); instead, each ABE uses an evolved deoxyadenosine deaminase domain-typically fused to the N-terminal end of the Cas9 nickase—to chemically modify an adenosine nucleoside on one DNA strand, which (in combination with nicking of the other strand) enables highly precise and efficient A→G transition mutations at the targeted site.

Phenylalanine hydroxylase (PAH) deficiency is the most common inherited defect in amino acid metabolism. Severe PAH deficiency, also termed classic phenylketonuria (PKU), results in profound elevations of blood phenylalanine (Phe) levels that, when untreated, cause neurotoxicity that manifests as impaired cognitive development and a host of irreversible neuropsychiatric impairments (Blau et al. 2010; Levy et al. 2018). There remains a substantial unmet medical need for patients with classic PKU. Although the liver is spared from toxicity, the PAH gene is largely expressed in hepatocytes, and correction of the primary genetic defect solely within the liver would in principle be curative in PKU patients (Grisch-Chan et al., 2019).

Clinical trials of liver-directed gene therapy to treat PKU via PAH replacement are underway, but the adeno-associated viral (AAV) vectors used for gene therapy have substantial limitations: the lack of genomic integration of the replacement gene can result in loss of therapeutic effect over time, especially in younger patients with active liver growth; the vectors induce immune responses that prevent re-administration of therapy; and pre-existing AAV antibodies limit the number of patients who can receive gene therapy (van Spronsen et al., 2021).

Out of the more than 1000 PAH variants that have been cataloged in patients (Regier and Greene, 2017), the 5 most common pathogenic variants linked to classic PKU are all transition mutations, specifically G→A or C→T variants on the sense or antisense strand (Hillert et al., 2020). As such, each of these variants is potentially amenable to correction by gene editing. For example, adenine base editing, which can engineer site-specific A→G changes on either DNA strand (Gaudelli et al., 2017), would be effective to correct each of these variants. The PAH c.842C>T (p.Pro281Leu) variant was focused on in this study because it is particularly amenable to correction by adenine base editing (as explained and demonstrated below). The PAH c.842C>T variant has its highest prevalence in populations in the Middle East, Russia, and Europe (Hillert et al., 2020) but is widespread. Patients homozygous for this variant do not respond at all to sapropterin (Leuders et al., 2014), limiting their treatment options.

Studies were performed demonstrating the viability of adenine base editing as a therapeutic approach in vivo. Most notably, lipid nanoparticles (LNPs) have proven effective at delivering an adenine base editor, encoded in mRNA, into the livers of non-human primates (Musunuru et al., 2021; Rothgangl et al., 2021). The adenine base editor efficiently introduced a loss-of-function variant into the PCSK9 cholesterol-regulating gene, achieving saturation editing of the hepatocytes in the liver and reducing the PCSK9 protein by ≈₉₀% without any adverse health consequences (Musunuru et al., 2021). In a recent clinical trial, LNP-mediated delivery of a nuclease editor (CRISPR-Cas9) into the liver to introduce loss-of-function mutations into a target gene (TTR) was safely tolerated and resulted in up to 96% reduction of the protein product (transthyretin) (Gillmore et al., 2021). Accordingly, a broad range of editing therapies can now be developed to ameliorate symptoms of a variety of diseases for which gene alterations in the liver would be curative.

The efficacy of ABEs to make targeted edits in the PCSK9 gene in primary human hepatocytes is described in Musunuru et al., 2021. In an effort to inactivate the PCSK9 gene, 20 gRNAs were identified that target protospacer DNA sequences positioned such that a PCSK9 splice-donor or splice-acceptor adenine lay within the activity window of ABE8.8. For each candidate target site, in vitro transcribed ABE8.8 messenger RNA (mRNA) along with a chemically synthesized gRNA were co-transfected into primary human hepatocytes. Varying degrees of base editing of the target adenine across the 20 gRNAs were observed (FIG. 1C), with the highest level of editing occurring with the gRNA targeting the splice-donor adenine at the boundary of PCSK9 exon 1 and intron 1, disruption of which results in premature truncation of the PCSK9 protein product. In further studies the same exon 1 splice-donor adenine in wild-type mice and in PCSK9-humanized mice in vivo was targeted, and, ultimately, in non-human primates in vivo, achieving saturation editing (i.e., editing of virtually all alleles in all hepatocytes in the liver) (Musunuru et al., 2021).

With PCSK9, the goal was to edit the wild-type gene, which is endogenous in primary human hepatocytes or cultured hepatocyte lines from any source. In contrast, with PAH, the goal was to correct a rare human mutation, c.842C>T (FIG. 2). However, there are no readily available primary human hepatocytes or cultured hepatocyte lines bearing that variant. Moreover, due to the limited ability of primary human hepatocytes to proliferate or to persist in culture more than a few days, there is no possibility of editing the variant into the hepatocytes to allow for subsequent testing of variant correction. Accordingly, a lentiviral platform was established that allows for simultaneous introduction of (1) a short stretch of the PAH genomic sequence bearing the c.842C>T variant, (2) a cassette encoding a gRNA matched to and intended to edit the c.842C>T variant, and (3) a cassette encoding an ABE of choice into the genomes of primary human hepatocytes. This permits the assessment of the efficiency of direct correction of the variant in the cells that most closely match the intended target cells of a human therapeutic, hepatocytes in vivo.

Using a lentiviral platform, two gRNAs were identified that target protospacer DNA sequences with NGG PAMs (matching the preference of S. pyogenes Cas9) that are positioned such that the PAH c.842C>T variant (i.e., the variant adenine on the antisense strand) lies within the activity window of ABE8.20, which has among the broadest windows of the eighth-generation ABEs (FIG. 2). These two protospacer DNA sequences are particularly attractive because each has a very high MIT specificity score (which summarizes all off-targets into a single number from 0-100), indicating a very high degree of orthogonality to other sequences in the human genome and, thus, a much lower likelihood of off-target editing.

Lentiviruses encoding the PAH c.842C>T genomic sequence (73 nucleotides flanking the variant), one of the PAH gRNAs, and ABE8.20 were generated. As a positive control, similar lentiviruses for the validated PCSK9 exon 1 splice-donor site (described above) were used. Primary human hepatocytes were obtained as cryo-frozen stocks from a commercial vendor. Twenty-four hours after replating of the hepatocytes, lentiviruses were added to the media for infection of the cells at three titers (low, middle, high). Three days later, genomic DNA was harvested from the cells and next-generation sequencing of PCR amplicons generated from the lentiviral-integrated PAH c.842C>T genomic sequence performed.

At the highest titer, the ABE/gRNA set achieved a high level of base editing proportion of the target adenine (which, upon conversion to guanine, corrects the PAH c.842C>T variant). While promising, we developed additional ABEs with narrower editing windows and/or 3′-shifted editing windows to preserve efficient editing of the target adenine and minimize bystander editing.

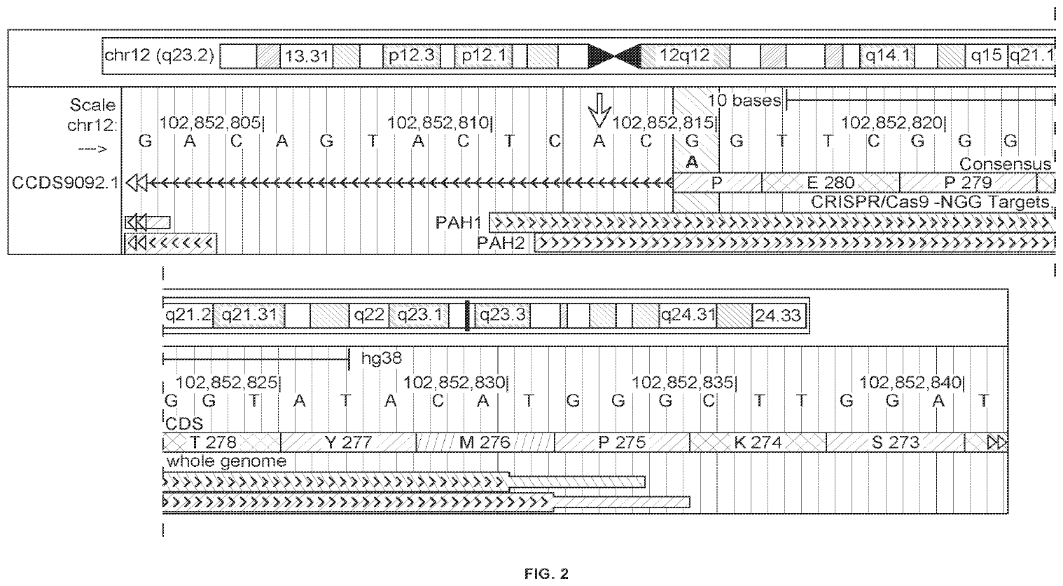

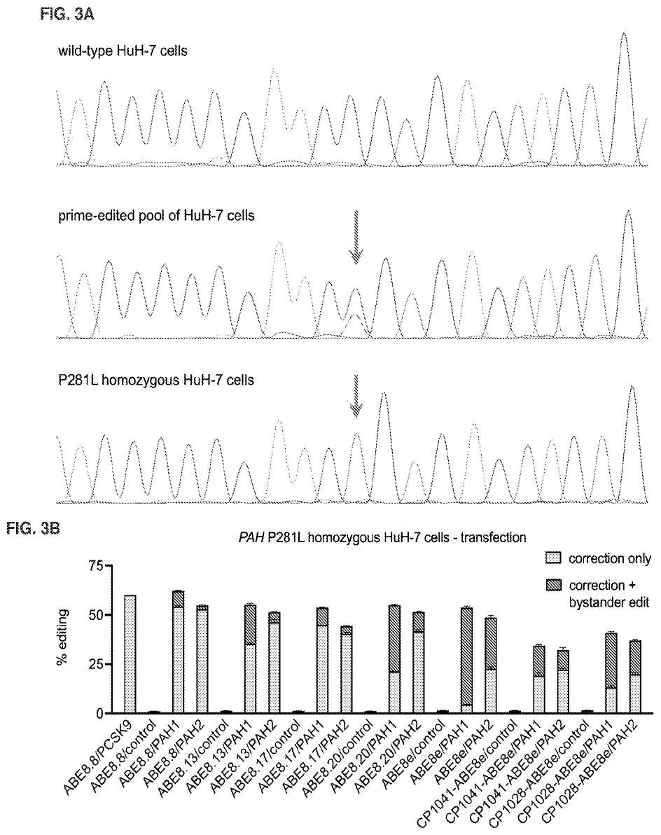

An model was generated comprising HuH-7 human hepatoma cells harboring the PAH c.842C>T variant. Because HuH-7 cells are hepatocyte-like cells, proliferate indefinitely in culture, are highly transfectable, and can undergo single-cell cloning to generate genetically modified cell lines, the PAH c.842C>T variant was introduced into HuH-7 cells via editing. Prime editing was utilized to make the desired edit. A recently reported prime editor configuration—PEmax paired with dnMLH1 (a dominant negative MLH1 that knocks down mismatch repair and thereby improves editing efficiency) along with an engineered prime editing gRNA (epegRNA) and a nicking gRNA (ngRNA) (Chen et al., 2021; Nelson et al., 2021) was introduced into HuH-7 cells via transient transfection of plasmids. A pool of cells in which a large proportion of the PAH alleles carry the PAH c.842C>T variant was obtained (FIG. 3A). A clonal cell line derived from the pooled cells, homozygous for the PAH c.842C>T variant, was isolated (FIG. 3A).

Developed by directed evolution of a seventh-generation ABE, ABE7.10, at least forty-one modified eighth-generation ABEs have been reported that all can have higher editing efficiencies compared to ABE7.10 in mammalian cells (Gaudelli et al., 2020; Richter et al., 2020). In addition, 30 inlaid base editors (IBEs) have been reported, several of which have higher editing efficiencies compared to the standard N-terminal deaminase-fused ABE, while having 3′-shifted editing windows (Chu et al., 2021). Of note, the possibility of using a SaCas9-containing ABE was considered. However, there is no SaCas9 NNGRRT PAM or SaCas9 KKH variant NNNRRT PAM that is properly positioned to place the target PAH c.842C>T adenine within the editing window (FIG. 2). Moreover, even if there had been an appropriately positioned PAM, SaCas9-containing ABEs have much broader editing windows than SpCas9-containing ABEs, making counterproductive bystander editing around the target adenine virtually unavoidable. As such, SpCas9-containing ABEs were utilized.

Using the clonal HuH-7 cell line homozygous for the PAH c.842C>T variant (homozygous P281L HuH-7 cells), generated with prime editing as described above, a variety of eighth-generation ABEs in combination with each of two gRNAs, PAH1 and PAH2 (SEQ ID NOS: 1 and 3) (FIG. 2) were tested for their ability to correct the variant (FIG. 3B). All of the ABE/gRNA sets corrected large proportions of the variant alleles to wild-type, but with highly varied levels of bystander editing. Among the ABE/gRNA sets tested, ABE8.8 displayed the most favorable balance of efficient variant correction and minimized bystander editing, either in combination with PAH1 or with PAH2. By way of comparison, ABE8.8 in combination with the previously validated gRNA targeting the human PCSK9 exon 1 splice-donor adenine effected 60% editing of the PCSK9 gene in the same cell line.

### Example 2

### Adenine Base Editing In Vivo Using Various Delivery Methods.

Three different delivery modalities were explored to achieve base editing of target genes in the mouse liver in vivo: adeno-associated viral (AAV) vectors, lipid nanoparticles (LNPs), and engineered virus-like particles (eVLPs). AAV vectors are well suited for delivery to hepatocytes in the liver, especially AAV serotype 8 (AAV8), but they have the limitation that they can accommodate only up to ≈4.7 kb of cargo. In light of the relatively large size of S. pyogenes Cas9 (SpCas9), a gene encoding a SpCas9 base editor is too large to fit into a single AAV vector along with a promoter, a polyadenylation sequence, and a gRNA expression cassette. Accordingly, a split-intein configuration has been used (Villiger et al., 2018; Levy et al., 2020) to deliver SpCas9 base editors in two halves on two AAV vectors, with spontaneous assembly of the two halves into a functional protein upon expression in hepatocytes. The smaller size of S. aureus Cas9 (SaCas9) can also be used to advantage to package SaCas9 base editors with gRNAs into all-in-one, single AAV vectors.

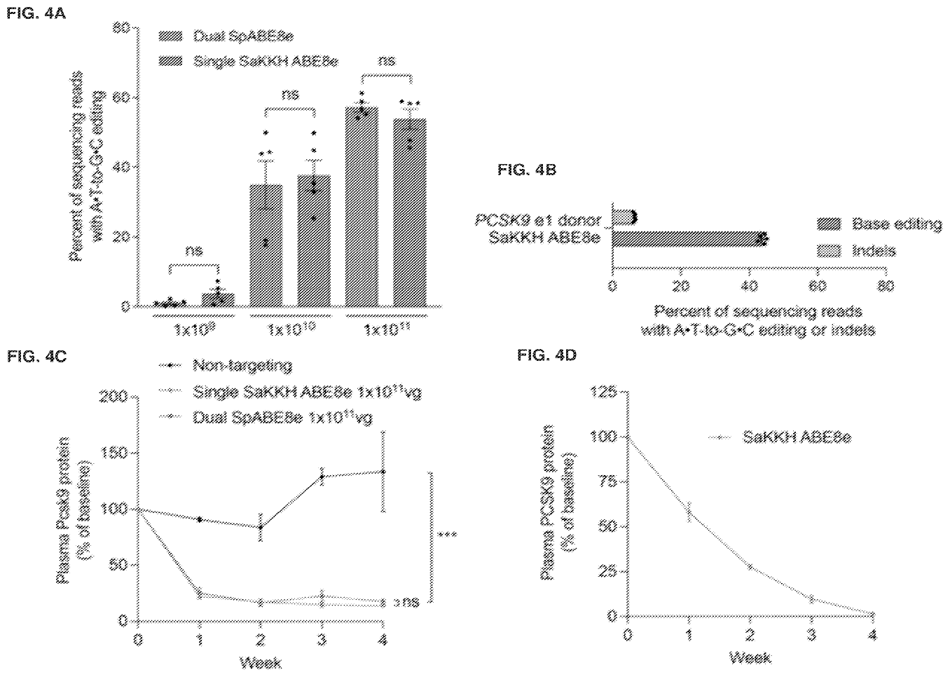

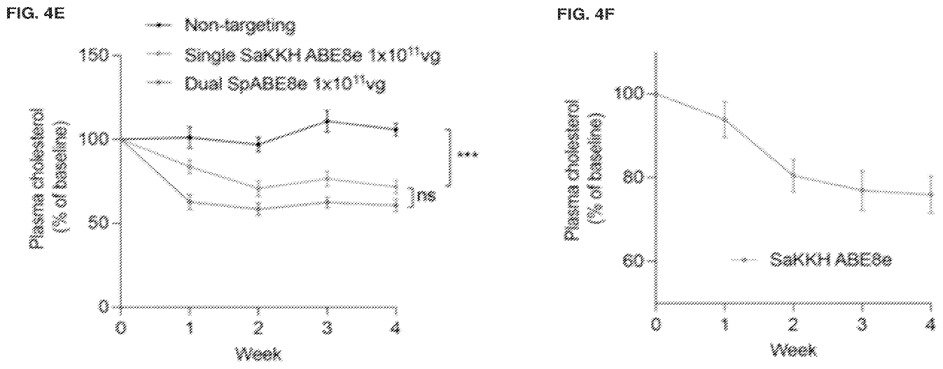

A dual AAV configuration encoding the standard SpCas9 version of ABE8e (SpABE8e) and a gRNA targeting the murine Pcsk9 exon 1 splice-donor adenine was employed, with the goal of knocking down Pcsk9 in the mouse liver. Single AAVs encoding an SaCas9 KKH variant (relaxed PAM)-containing version of ABE8e (SaKKH ABE8e) were also employed with either a gRNA targeting the murine Pcsk9 exon 1 splice-donor adenine—intended for wild-type mice—or a gRNA targeting the human PCSK9 exon 1 splice-donor adenine—intended for PCSK9-humanized mice. The humanized mice have complete knockout of the endogenous mouse Pcsk9 gene as well as a bacterial artificial chromosome transgene harboring the entirety of the human PCSK9 locus (Essalmani et al., 2018). The AAV vectors were administered to 6- to 8-week-old mice systemically via retro-orbital injection at various doses (Davis et al., 2022). Upon necropsy 4 weeks after AAV treatment, similar levels of whole-liver base editing with dual-AAV SpABE8e and single-AAV SaKKH ABE8e were observed, with ≈60% base editing observed at the highest dose (FIG. 4A). Notably, given that hepatocytes constitute 60%-70% of the cells in the liver, that level of editing in the whole liver approaches saturation editing of the hepatocytes. Single-AAV SaKKH ABE8e also produced a high level of whole-liver editing in PCSK9-humanized mice (FIG. 4B). The editing in both wild-type mice and PCSK9-humanized mice was accompanied by near-complete knockdown of blood PCSK9 protein levels (FIG. 4C, FIG. 4D) and substantial reduction of blood cholesterol levels (FIG. 4E, FIG. 4F), with no significant differences observed in the effects of dual-AAV-mediated versus single-AAV-mediated editing.

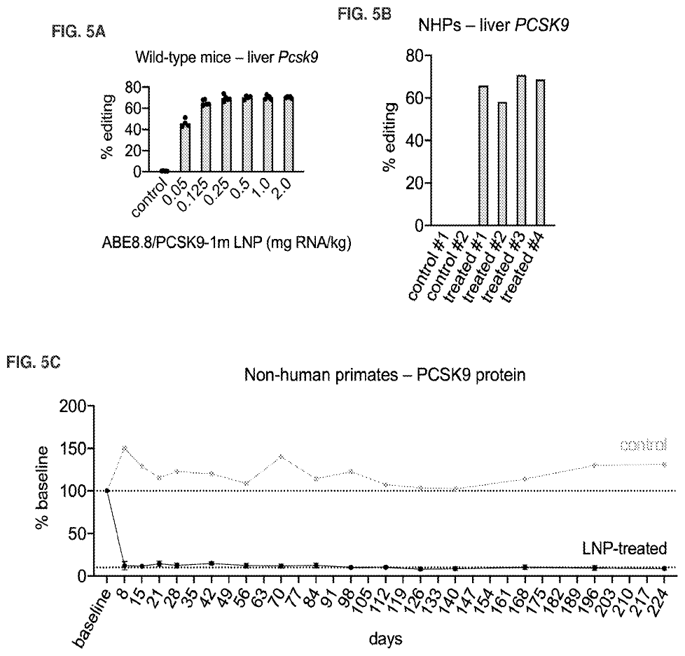

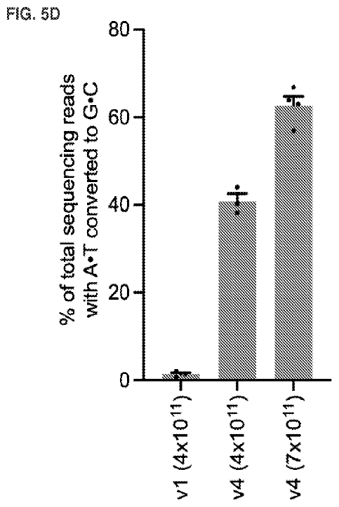

LNPs offer the advantage of transient expression and activity of the base editor via mRNA and gRNA, since the RNA molecules are short-lived; moreover, the in vitro transcribed mRNA component does not have an intrinsic size limitation and can readily accommodate even the largest base editors. LNPs were formulated containing ABE8.8 mRNA and the gRNA targeting the murine Pcsk9 exon 1 splice-donor adenine, at a 1:1 ratio by weight, and administered to wild-type mice via intravenous infusion at a range of doses (Musunuru et al., 2021). Upon necropsy 1 week after LNP infusion, 60%-70% whole-liver base editing at various doses down to 0.125 mg/kg body weight was observed (FIG. 5A), consistent with saturation editing of the hepatocytes in the liver. Subsequent studies using similar LNP formulations demonstrated saturation editing in the livers of non-human primates, with profound reductions in blood PCSK9 protein levels that were stable to at least 8 months (FIG. 5B, FIG. 5C) (Musunuru et al., 2021). The LNP treatment was associated with only mild transient elevations in AST and ALT that resolved within a few days. These results forecast the viability of LNP base-editing therapies in human patients. eVLPs also offer the advantage of transient expression and activity of the base editor via protein and gRNA, since the ribonucleoproteins are short-lived. eVLPs were used to deliver SpABE8e and the gRNA targeting the murine Pcsk9 exon 1 splice-donor adenine into wild-type mice at various doses, reducing serum Pcsk9 levels up to 78% following up to 63% liver editing (FIG. 5D) (Banskota et al., 2022).

### Example 3

Generating Two Humanized Mouse Models with the Human PAH c.842C>T Variant.

In order to assess for editing activity of the prioritized ABE/gRNA set(s) in hepatocytes in vivo, an animal model that harbors not only the PAH c.842C>T variant but also the protospacer DNA sequence and the surrounding sequence context that allows for a functional readout of variant correction must be generated. Accordingly, humanized mouse models in which a portion of the endogenous mouse Pah locus has been replaced with the orthologous portion of the human PAH locus containing the variant have been created. This degree of humanization facilitates assessment of the therapeutic effect of base editing of the PAH c.842C>T variant via disease-relevant phenotypic readouts.

A humanized mouse model can be generated using a number of approaches. In one approach, the mouse exon containing the site of the PAH c.842C>T variant (exon 7) was replaced as well as the surrounding introns (intron 6 and intron 7) and the flanking exons (exon 6 and exon 8) (FIG. 6). This was achieved via electroporation of a PAH targeting vector into mouse embryonic stem cells and generation of chimeras, followed by breeding, in the inbred C57BL/6J background. Homozygous mice, when maintained on a normal chow diet, displayed signs of PKU including elevated blood L-phenylalanine levels, growth retardation, and hypopigmentation (agouti instead of black fur). The humanized PAH c.842C>T variant can be maintained in the heterozygous state, with heterozygous mice being entirely healthy.

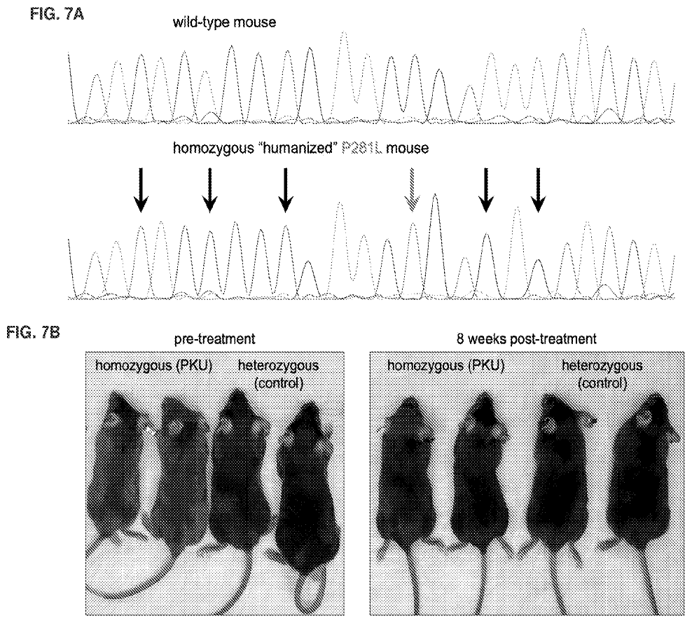

In another approach, we used CRISPR-Cas9 targeting in mouse embryos to generate a minimally humanized PKU model, in the C57BL/6J background, in which we replaced a small portion of the endogenous mouse Pah exon 7 with the orthologous human sequence spanning the PAH1 and PAH2 protospacers and containing the c.842C>T variant (FIG. 7A). Homozygous mice had phenotypes consistent with PKU (FIG. 7B).

### Example 4

### ABE Activity in Human Hepatocytes Via LNP Delivery.

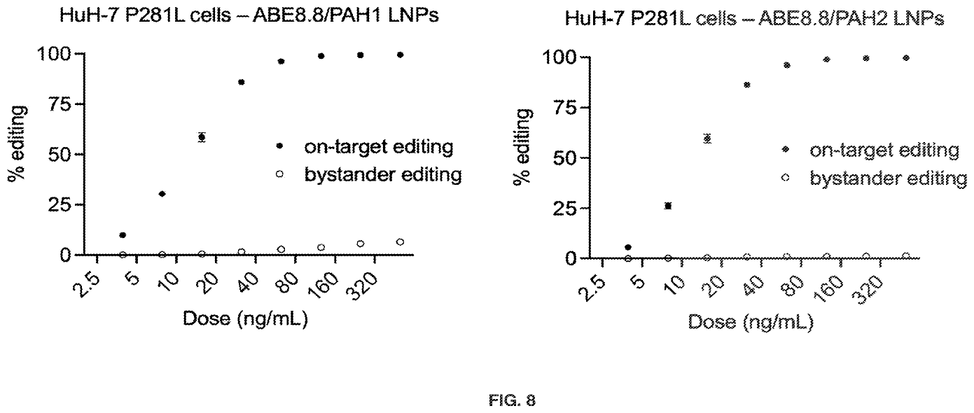

For LNP delivery, the PAH1 and PAH2 gRNAs with appropriately positioned 2′-O-methyl and phosphorothioate modifications were synthesized by Agilent. mRNA encoding ABE8.8 was generated via in vitro transcription and purification by the University of Pennsylvania Engineered mRNA and Targeted Nanomedicine Core. The Core also formulated the mRNA with the gRNA into LNPs containing standard lipid components (ionizable cationic lipid, 1,2-distearoyl-sn-glycero-3-phosphocholine, cholesterol, and a PEG-lipid). We performed dose-response studies with the LNPs using the homozygous P281L HuH-7 cell line (FIG. 8); there was essentially 100% corrective editing at higher doses, with virtually identical EC₅₀ values for the two gRNAs, establishing equivalent potency.

### Example 5

### Using ONE-Seq and ABE-Digenome-Seq to Evaluate Off-Target Editing.

Two complementary techniques can be employed to identify candidate genome-wide off-target sites, which are then evaluated in primary hepatocytes for off-target editing. In the first technique, termed OligoNucleotide Enrichment and sequencing (ONE-seq) (Petri et al., 2021), a synthetic library of oligonucleotides encoding genomic sites with a high degree of homology to the gRNA's protospacer DNA sequence (e.g., all sites that have up to 6 mismatches with the protospacer, and all sites that have up to 4 mismatches and up to 2 bulges compared to the protospacer) are contacted with an ABE/gRNA set in vitro and sites which undergo base editing identified. The second technique is an ABE-adapted version of Digenome-seq (Liang et al., 2019; Kim et al., 2019), an unbiased approach in which genomic DNA isolated from primary human hepatocytes is contacted with an ABE/gRNA set in vitro and determine which sites undergo base editing. In both ONE-seq and ABE-Digenome-seq, the ABE converts an adenosine nucleoside on one strand into inosine, and also nicks the other strand; treatment with EndoV enzyme specifically cleaves a DNA strand near an inosine nucleoside, which when combined with the nicking on the other strand yields the equivalent of a double-strand break. End repair, adaptor ligation, PCR amplification, and next-generation sequencing then determine the frequency at which each site underwent editing in vitro (if at all), resulting in a rank-ordered list of candidate off-target sites.

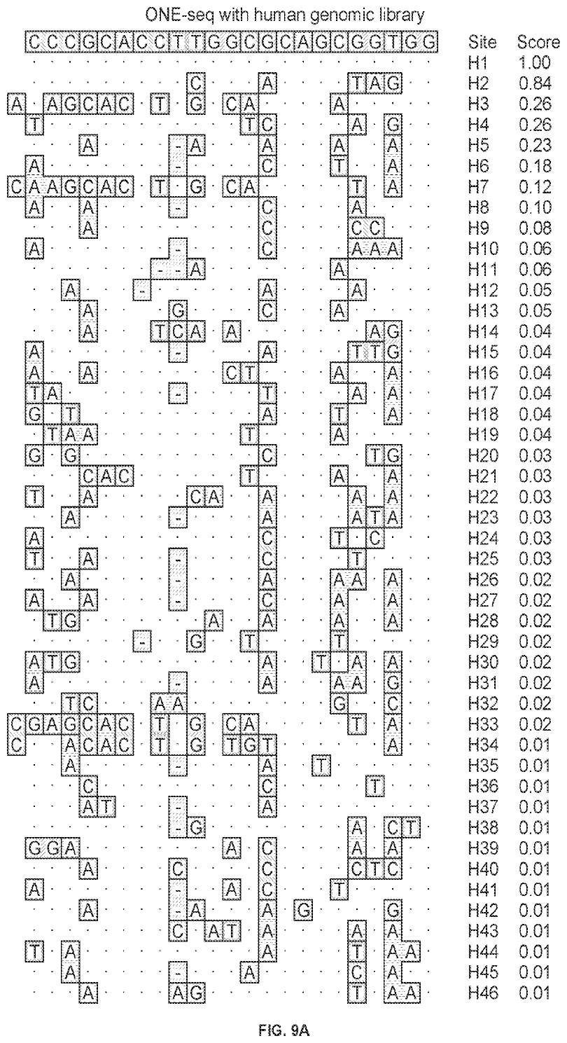

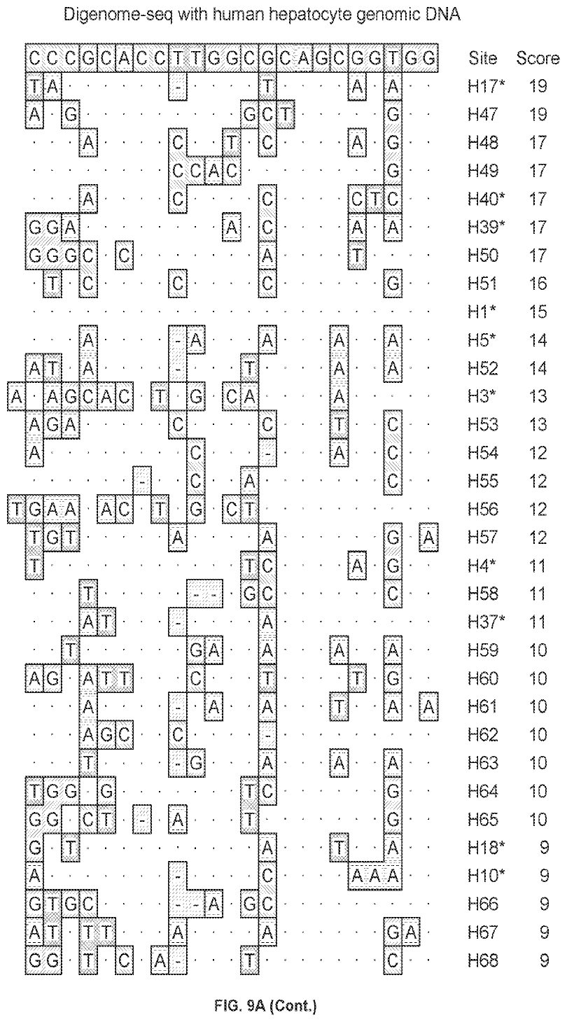

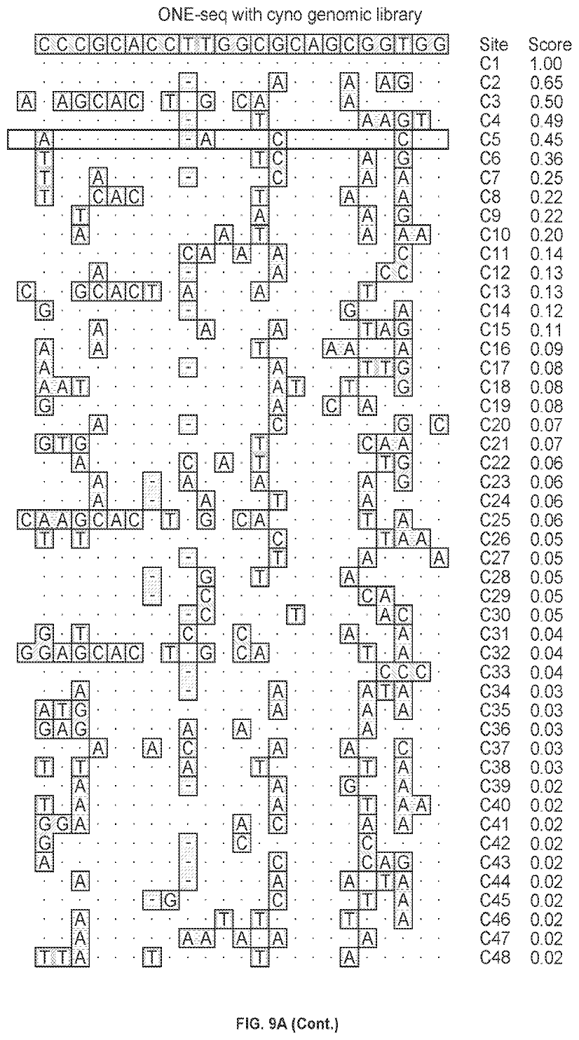

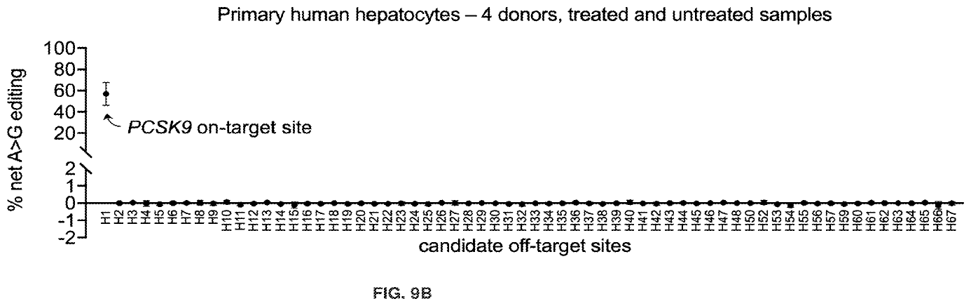

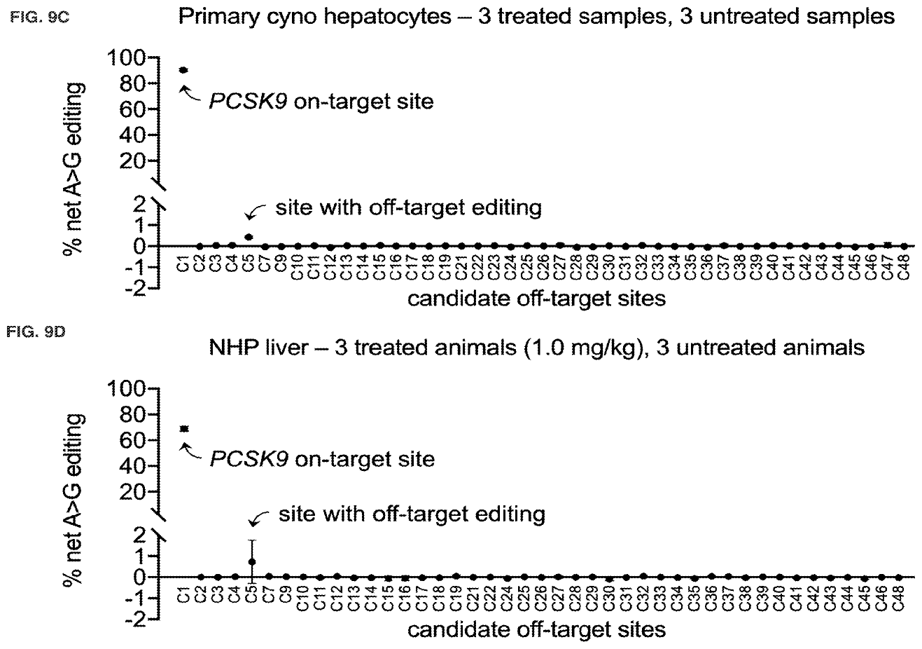

To evaluate off-target editing mediated by ABE8.8 and a PCSK9 gRNA in primary human hepatocytes, (1) ONE-seq with a synthetic human genomic library that was selected by homology to the PCSK9 gRNA protospacer DNA sequence and (2) ABE-Digenome-seq using whole-genome sequencing of human hepatocyte genomic DNA treated with ABE8.8 protein and the PCSK9 gRNA (Musunuru et al., 2021) were performed and the top 46 ONE-seq-nominated sites and the top 33 ABE-Digenome-seq-nominated sites (10 sites were common to both lists) in LNP-treated versus untreated hepatocytes from four individual donors assessed (FIG. 9A). By next-generation sequencing of targeted PCR amplicons, there was discernible editing only at the PCSK9 target site (FIG. 9B).

Off-target editing mediated by ABE8.8 and the PCSK9 gRNA in primary cynomolgus monkey hepatocytes was also evaluated by performing ONE-seq with a synthetic cynomolgus monkey genomic library that was selected by homology to the PCSK9 gRNA protospacer DNA sequence. The top 48 ONE-seq-nominated sites (of which the PCSK9 target site was the top site) were assessed using next-generation sequencing of targeted PCR amplicons from LNP-treated versus untreated samples (FIG. 9A). In LNP-treated primary cynomolgus monkey hepatocytes, besides editing at the PCSK9 target site there was off-target editing (mean of <1%) that was evident at only one site (designated C5), which has poor homology to the human genome (FIG. 9C). Assessing the same 48 sites in liver samples from monkeys that were treated with LNPs at a dose of 1.0 mg/kg, off-target editing at a low level was again observed (mean of <1%) only at the C5 site (FIG. 9C). The concordance of the results relating to off-target editing in primary cynomolgus monkey hepatocytes in vitro and monkey liver in vivo suggests that primary hepatocytes are an appropriate model for in vivo liver editing.

We have performed ONE-seq with ABE8.8 in combination with either the PAH1 gRNA or PAH2 gRNA used to correct the PAH c.842C>T variant. We assessed the top ≈150 ABE8.8/PAH1 ONE-seq-nominated sites with next-generation sequencing of targeted PCR amplicons from ABE8.8/PAH1 plasmid-transfected versus control-transfected homozygous P281L HuH-7 genomic DNA samples. We observed two sites with low-level off-target base editing (≈0.1% and ≈0.2%) and two sites with higher off-target base editing (≈7% and ≈1%) (FIG. 10A). We also assessed the top ≈50 ABE8.8/PAH2 ONE-seq-nominated sites with next-generation sequencing of targeted PCR amplicons from ABE8.8/PAH2 plasmid-transfected versus control-transfected homozygous P281L HuH-7 genomic DNA samples. We observed a site with low-level off-target base editing (≈0.2%) and a site with substantial off-target base editing (≈=8%) (FIG. 10B).

### Example 6

A Lentiviral Platform to Assess Editing with a Library of Genomic Sites.

A limitation of all existing off-target methods is that each is tied to the specific individual genome represented by the cells or the genomic DNA sample used for analysis. Even assessing several different samples from different people does not begin to capture the full scope of human genetic diversity. ONE-seq provides one possible solution to this problem, because it uses a synthetic oligonucleotide library that, in principle, could include genomic sequences that account for common and rare variation cataloged in human populations. However, if ONE-seq were to identify any variant sequences as highly ranked candidate off-target sites—especially sequences with rare variants—it would not be possible to procure primary human hepatocytes bearing those variant sequences, preventing a direct evaluation of off-target editing in the target cell type.

To overcome this limitation, lentiviruses can be used to insert a library of variant sequences into the genomes of primary human hepatocytes, followed by assessment of editing of those variant sequences by an ABE/gRNA set.

We have developed Lenti-seq, which unlike ONE-seq and Digenome-seq (and uniquely among the assays that can directly assess ABE editing) is a cellular-based assay. Lenti-seq is an in cellulīs version of ONE-seq, in which a lentiviral pool is used to introduce a barcoded ONE-seq library into the genomes of hepatocytes. The hepatocytes are treated with ABE/gRNA LNPs; very deep next-generation sequencing of the lentiviral cassette harboring the oligonucleotides, followed by barcode deconvolution, identifies any sequences with detectable off-target editing. Lenti-seq can be performed with primary human hepatocytes from multiple donors, HuH-7 cells, and other cell types. As a proof of concept of Lenti-seq, we made a lentiviral library harboring the top ≈200 ABE8.8/PAH2 ONE-seq-nominated sites, infected HuH-7 cells with the lentiviral library, and then transduced the cells with ABE8.8/PAH2 LNPs. We observed substantial editing of the exogenously introduced PAH on-target site as well as an single off-target site (FIG. 10C).

### Example 7

gRNA Selection and Optimization.

Two gRNAs were synthesized that target protospacer DNA sequences with NGG PAMs for which the PAH c.842C>T variant adenine is within the editing window of most ABEs (FIG. 2). Notably, one of the gRNAs (PAH1) in its wild-type version has an MIT specificity score of 98, and the other (PAH2) in its wild-type version has a score of 95 (on a scale of 0 to 100, with a higher number predicting fewer off-target effects). By way of comparison, the previously validated PCSK9 gRNA described above, with no detectable off-target editing in primary human hepatocytes, has an MIT specificity score of 90. Thus, both of the PAH gRNAs in the versions that match the PAH c.842C>T variant are expected to have minimal or no detectable off-target editing. SEQ ID NOS: 1 and 3 are exemplary guide polynucleotides. SEQ ID NOS: 2 and 4 are the spacer sequences for SEQ ID NOS: 1 and 3 respectively. SEQ ID NOS: 5 and 6 are PAH encoding nucleic acids with the c.842C>T variant that are complementary to and targeted by the spacer sequences.

In order to reduce off-target editing while preserving efficient on-target editing (Donohoue et al., 2021), we made a series of 28 hybrid gRNAs (SEQ ID NOS: 16-44) based on PAH1 in which certain positions in the spacer sequence were variously substituted with DNA nucleotides instead of RNA nucleotides, while preserving complementarity to the target PAH c.842C>T sequence (SEQ ID NO: 5). We screened all 28 hybrid gRNAs, along with the original PAH1 gRNA, via transfection of ABE8.8 mRNA and gRNA into homozygous P281L HuH-7 cells, for editing of the on-target site and of the off-target site identified to have the highest level of off-target editing for PAH1 (FIG. 10A). We found that two hybrid gRNAs (PAH1_hyb26 and PAH1_hyb27) performed the best (Tables 1-4). In interrogating all four of the off-target editing sites identified for PAH1 (FIG. 10A), we found that PAH1_hyb26 and PAH1_hyb27 each eliminated all detectable off-target editing (i.e., editing greater than 0.10%, the lower limit of detection) while retaining full on-target editing activity.

TABLE 1

| Guide | SEQ | | DNA | Total | PAH on- | Off-target |
|---|---|---|---|---|---|---|
| polynucleotide | ID NO | Spacer sequence* | position(s) | mRNA (ng) | target^{†} | site #3^{†} |
| PAH1 | 2 | UCACAGUUCGGGGGUAUACA | — | 1000 | 79.6 | 0.90 |
| | | | | 340 | 63.2 | 0.28 |
| | | | | 120 | 32.3 | 0.11 |
| PAH1_hyb1 | 112 | UCACAGUUCGGGGGUAUACA | 3 | 1000 | 89.4 | 0.57 |
| | | | | 340 | 56.4 | 0.33 |
| | | | | 120 | 38.0 | 0.01 |
| PAH1_hyb2 | 13 | UCACAGUUCGGGGGUAUACA | 4 | 1000 | 90.9 | 0.22 |
| | | | | 340 | 75.9 | 0.05 |
| | | | | 120 | 36.6 | 0.03 |
| PAH1_hyb3 | 14 | UCACAGUUCGGGGGUAUACA | 5 | 1000 | 82.3 | 0.25 |
| | | | | 340 | 63.1 | 0.16 |
| | | | | 120 | 30.9 | 0.00 |
| PAH1_hyb4 | 15 | UCACAGUUCGGGGGUAUACA | 6 | 1000 | 84.7 | 0.40 |
| | | | | 340 | 65.1 | 0.19 |
| | | | | 120 | 32.4 | 0.16 |
| PAH1_hyb5 | 16 | UCACAGTUCGGGGGUAUACA | 7 | 1000 | 91.9 | 1.27 |
| | | | | 340 | 72.4 | 0.52 |
| | | | | 120 | 33.3 | 0.10 |
| PAH1_hyb6 | 17 | UCACAGUTCGGGGGUAUACA | 8 | 1000 | 90.5 | 1.68 |
| | | | | 340 | 63.5 | 0.51 |
| | | | | 120 | 27.5 | 0.18 |
| PAH1_hyb7 | 18 | UCACAGUUCGGGGGUAUACA | 9 | 1000 | 86.5 | 0.46 |
| | | | | 340 | 69.6 | 0.25 |
| | | | | 120 | 39.6 | 0.16 |
| PAH1_hyb8 | 19 | UCACAGUUCGGGGGUAUACA | 10 | 1000 | 81.4 | 0.35 |
| | | | | 340 | 63.5 | 0.11 |
| | | | | 120 | 32.7 | 0.00 |
| PAH1_hyb9 | 20 | UCACAGUUCGGGGGUAUACA | 3, 4 | 1000 | 86.6 | 0.08 |
| | | | | 340 | 69.7 | −0.01 |
| | | | | 120 | 33.6 | 0.04 |
| PAH1_hyb10 | 21 | UCACAGUUCGGGGGUAUACA | 4, 5 | 1000 | 84.6 | 0.12 |
| | | | | 340 | 65.6 | 0.00 |
| | | | | 120 | 30.3 | 0.00 |
| PAH1_hyb11 | 22 | UCACAGUUCGGGGGUAUACA | 5, 6 | 1000 | 77.9 | 0.21 |
| | | | | 340 | 56.2 | 0.06 |
| | | | | 120 | 28.4 | 0.01 |
| PAH1_hyb12 | 23 | UCACAGTUCGGGGGUAUACA | 6, 7 | 1000 | 85.7 | 0.77 |
| | | | | 340 | 63.0 | 0.42 |
| | | | | 120 | 33.4 | 0.12 |
| PAH1_hyb13 | 24 | UCACAGTTCGGGGGUAUACA | 7, 8 | 1000 | 89.6 | 2.21 |
| | | | | 340 | 62.3 | 0.88 |
| | | | | 120 | 30.5 | 0.20 |
| PAH1_hyb14 | 25 | UCACAGUTCGGGGGUAUACA | 8, 9 | 1000 | 89.1 | 0.90 |
| | | | | 340 | 61.8 | 0.27 |
| | | | | 120 | 30.6 | 0.06 |

*The bold, underlined nucleotides indicate DNA nucleotide substitutions
^{†}The numbers indicate net % base editing at the site (compared to control background)

TABLE 2

| Guide | SEQ | | DNA | Total | PAH on- | Off-target |
|---|---|---|---|---|---|---|
| polynucleotide | ID NO | Spacer sequence* | position(s) | mRNA (ng) | target^{†} | site #3^{†} |
| PAH1 | 2 | UCACAGUUCGGGGGUAUACA | — | 1000 | 91.6 | 1.32 |
| | | | | 340 | 79.6 | 0.66 |
| | | | | 120 | 44.3 | 0.26 |
| PAH1_hyb15 | 26 | UCACAGUUCGGGGGUAUACA | 9, 10 | 1000 | 93.9 | 0.44 |
| | | | | 340 | 82.6 | 0.17 |
| | | | | 120 | 51.9 | 0.08 |
| PAH1_hyb16 | 27 | UCACAGUUCGGGGGUAUACA | 3, 4, 5 | 1000 | 93.4 | 0.24 |
| | | | | 340 | 80.0 | 0.04 |
| | | | | 120 | 46.0 | 0.05 |
| PAH1_hyb17 | 28 | UCACAGUUCGGGGGUAUACA | 4, 5, 6 | 1000 | 89.0 | 0.10 |
| | | | | 340 | 81.7 | 0.06 |
| | | | | 120 | 44.1 | 0.02 |
| PAH1_hyb18 | 29 | UCACAGTUCGGGGGUAUACA | 5, 6, 7 | 1000 | 93.6 | 0.29 |
| | | | | 340 | 79.6 | 0.20 |
| | | | | 120 | 49.2 | 0.08 |
| PAH1_hyb19 | 30 | UCACAGTTCGGGGGUAUACA | 6, 7, 8 | 1000 | 93.3 | 2.04 |
| | | | | 340 | 85.1 | 1.28 |
| | | | | 120 | 43.7 | 0.42 |
| PAH1_hyb20 | 31 | UCACAGTTCGGGGGUAUACA | 7, 8, 9 | 1000 | 92.4 | 2.85 |
| | | | | 340 | 80.6 | 1.40 |
| | | | | 120 | 44.1 | 0.46 |
| PAH1_hyb21 | 32 | UCACAGUTCGGGGGUAUACA | 9, 8, 10 | 1000 | 88.2 | 0.42 |
| | | | | 340 | 74.9 | 0.15 |
| | | | | 120 | 41.0 | 0.10 |

*The bold, underlined nucleotides indicate DNA nucleotide substitutions
^{†}The numbers indicate net % base editing at the site (compared to control background)

TABLE 3

| Guide | SEQ | | DNA | Total | PAH on- | Off-target |
|---|---|---|---|---|---|---|
| polynucleotide | ID NO | Spacer sequence | position(s) | mRNA (ng) | target^{†} | site #3^{†} |
| PAH1 | 2 | UCACAGUUCGGGGGUAUACA | — | 1000 | 89.4 | 1.96 |
| | | | | 340 | 69.6 | 0.86 |
| | | | | 120 | 40.4 | 0.28 |
| PAH1_hyb22 | 33 | UCACAGUUCGGGGGUAUACA | 3, 4, 5, 6 | 1000 | 86.5 | 0.09 |
| | | | | 340 | 72.1 | 0.04 |
| | | | | 120 | 37.2 | 0.02 |
| PAH1_hyb23 | 34 | UCACAGUUCGGGGGUAUACA | 3, 4, 9, 10 | 1000 | 45.6 | 0.04 |
| | | | | 340 | 73.7 | 0.01 |
| | | | | 120 | 41.9 | 0.03 |
| PAH1_hyb24 | 35 | UCACAGUUCGGGGGUAUACA | 4, 5, 9, 10 | 1000 | 89.7 | 0.02 |
| | | | | 340 | 74.2 | 0.02 |
| | | | | 120 | 41.5 | 0.03 |
| PAH1_hyb25 | 36 | UCACAGUUCGGGGGUAUACA | 5, 6, 9, 10 | 1000 | 91.9 | 0.01 |
| | | | | 340 | 77.4 | 0.03 |
| | | | | 120 | 41.0 | 0.01 |
| PAH1_hyb26 | 37 | UCACAGUUCGGGGGUAUACA | 3, 4, 5, 9, | 1000 | 93.3 | −0.02 |
| | | | 10 | 340 | 73.3 | 0.05 |
| | | | | 120 | 41.2 | 0.04 |
| PAH1_hyb27 | 38 | UCACAGUUCGGGGGUAUACA | 4, 5, 6, 9, | 1000 | 89.3 | −0.01 |
| | | | 10 | 340 | 69.5 | −0.01 |
| | | | | 120 | 37.6 | 0.04 |
| PAH1_hyb28 | 39 | UCACAGUUCGGGGGUAUACA | 3, 4, 5, 6, | 1000 | 83.2 | −0.01 |
| | | | 9, 10 | 340 | 64.5 | 0.03 |
| | | | | 120 | 35.9 | 0.01 |

*The bold, underlined nucleotides indicate DNA nucleotide substitutions
^{†}The numbers indicate net % base editing at the site (compared to control background)

TABLE 4

| Guide | | | | | | | | | |
|---|---|---|---|---|---|---|---|---|---|
| poly- | SEQ | | | Total | PAH | Off- | Off- | Off- | Off- |
| nucleo- | ID | | DNA | mRNA | on- | target | target | target | target |
| tide | NO | Spacer sequence* | position(s) | (ng) | target^{†} | site #1^{†} | site #2^{†} | site #3^{†} | site #4^{†} |
| PAH1 | 2 | UCACAGUUCGGGGGUAU | — | 1000 | 88.9 | 0.15 | 0.21 | 2.00 | 0.92 |
| | | ACA | | 340 | 71.4 | 0.17 | 0.10 | 1.04 | 0.44 |
| | | | | 120 | 38.3 | 0.03 | 0.08 | 0.28 | 0.04 |
| PAH1_ | 37 | UCACAGUUCGGGGGUAU | 3, 4, 5, | 1000 | 90.5 | 0.01 | 0.00 | 0.01 | 0.02 |
| hyb26 | | ACA | 9, 10 | 340 | 73.8 | 0.03 | 0.00 | 0.02 | −0.02 |
| | | | | 120 | 39.3 | 0.01 | 0.00 | 0.04 | −0.05 |
| PAH1_ | 38 | UCACAGUUCGGGGGUAU | 4, 5, 6, | 1000 | 89.3 | 0.02 | 0.01 | 0.00 | 0.02 |
| hyb27 | | ACA | 9, 10 | 340 | 69.6 | 0.07 | 0.10 | 0.04 | 0.06 |
| | | | | 120 | 38.3 | 0.03 | 0.00 | 0.01 | −0.05 |

*The bold, underlined nucleotides indicate DNA nucleotide substitutions
^{†}The numbers indicate net % base editing at the site (compared to control background)

The spacer sequences present in Tables 1-4 may be incorporated into guide RNA sequences with the structures present in Table 5. More specifically, the first 20 nucleotides (labeled N₂₀ Of mN*₃N₁₇ in the generic structures) can be replaced with SEQ ID NOs: 12-39. The sequences for the full hybrid oligonucleotide sequences with the N₂₀ replaced with SEQ ID NOs: 12-39 are identified as SEQ ID NOs: 41-68 respectively. The sequences for the lightly modified oligonucleotide sequence with the mN*₃N₁₇ replaced with SEQ ID NOs: 12-39 are identified as SEQ ID NOs: 72-99 respectively. The sequences for heavily modified oligonucleotide sequences with the mN*₃N₁₇ replaced with SEQ ID NOs: 12-39 are identified as SEQ ID NOs: 103-130 respectively.

TABLE 5

| | | |
|---|---|---|
| Spacer | SEQ ID NO | Guide oligonucleotide sequence |

Unmodified guide oligonucleotides

| | | |
|---|---|---|
| Generic | 40 | N₂₀GUUUUAGAGCUAGAAAUAGCAAGUUAAAAUAAGGCUAGUCCGUUAUCAAC |
| sequence | | UUGAAAAAGUGGCACCGAGUCGGUGCUUUU |
| PAH1 | 1 | UCACAGUUCGGGGGUAUACAGUUUUAGAGCUAGAAAUAGCAAGUUAAAAUAA |
| | | GGCUAGUCCGUUAUCAACUUGAAAAAGUGGCACCGAGUCGGUGCUUUU |
| PAH2 | 3 | CACAGUUCGGGGGUAUACAUGUUUUAGAGCUAGAAAUAGCAAGUUAAAAUAA |
| | | GGCUAGUCCGUUAUCAACUUGAAAAAGUGGCACCGAGUCGGUGCUUUU |
| PAH1_hyb1 | 41 | UCdACAGUUCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb2 | 42 | UCAdCAGUUCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb3 | 43 | UCACdAGUUCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb4 | 44 | UCACAdGUUCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb5 | 45 | UCACAGdTUCGGGGGUAUACAGUUUUAGAGCUAGAAAUAGCAAGUUAAAAUAA |
| | | GGCUAGUCCGUUAUCAACUUGAAAAAGUGGCACCGAGUCGGUGCUUUU |
| PAH1_hyb6 | 46 | UCACAGUdTCGGGGGUAUACAGUUUUAGAGCUAGAAAUAGCAAGUUAAAAUAA |
| | | GGCUAGUCCGUUAUCAACUUGAAAAAGUGGCACCGAGUCGGUGCUUUU |
| PAH1_hyb7 | 47 | UCACAGUUdCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb8 | 48 | UCACAGUUCdGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb9 | 49 | UCdAdCAGUUCGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb10 | 50 | UCAdCdAGUUCGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb11 | 51 | UCACdAdGUUCGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb12 | 52 | UCACAdGdTUCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb13 | 53 | UCACAGdTdTCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb14 | 54 | UCACAGUdTdCGGGGGUAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| PAH1_hyb15 | 55 | UCACAGUUdCdGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb16 | 56 | UCdAdCdAGUUCGGGGGUAUACAGUUUUAGAGCUAGAAAUAGCAAGUUAAAA |
| | | UAAGGCUAGUCCGUUAUCAACUUGAAAAAGUGGCACCGAGUCGGUGCUUUU |
| PAH1_hyb17 | 57 | UCAdCdAdGUUCGGGGGUAUACAGUUUUAGAGCUAGAAAUAGCAAGUUAAAA |
| | | UAAGGCUAGUCCGUUAUCAACUUGAAAAAGUGGCACCGAGUCGGUGCUUUU |
| PAH1_hyb18 | 58 | UCACdAdGdTUCGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb19 | 59 | UCACAdGdTdTCGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb20 | 60 | UCACAGdTdTdCGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb21 | 61 | UCACAGUdTdCdGGGGGUAUACAGUUUUAGAGCUAGAAAUAGCAAGUUAAAAU |
| | | AAGGCUAGUCCGUUAUCAACUUGAAAAAGUGGCACCGAGUCGGUGCUUUU |
| PAH1_hyb22 | 62 | UCdAdCdAdGUUCGGGGGUAUACAGUUUUAGAGCUAGAAAUAGCAAGUUAAAA |
| | | UAAGGCUAGUCCGUUAUCAACUUGAAAAAGUGGCACCGAGUCGGUGCUUUU |
| PAH1_hyb23 | 63 | UCdAdCAGUUdCdGGGGGUAUACAGUUUUAGAGCUAGAAAUAGCAAGUUAAAA |
| | | UAAGGCUAGUCCGUUAUCAACUUGAAAAAGUGGCACCGAGUCGGUGCUUUU |
| PAH1_hyb24 | 64 | UCAdCdAGUUdCdGGGGGUAUACAGUUUUAGAGCUAGAAAUAGCAAGUUAAAA |
| | | UAAGGCUAGUCCGUUAUCAACUUGAAAAAGUGGCACCGAGUCGGUGCUUUU |
| PAH1_hyb25 | 65 | UCACdAdGUUdCdGGGGGUAUACAGUUUUAGAGCUAGAAAUAGCAAGUUAAAA |
| | | UAAGGCUAGUCCGUUAUCAACUUGAAAAAGUGGCACCGAGUCGGUGCUUUU |
| PAH1_hyb26 | 66 | UCdAdCdAGUUdCdGGGGGUAUACAGUUUUAGAGCUAGAAAUAGCAAGUUAAA |
| | | AUAAGGCUAGUCCGUUAUCAACUUGAAAAAGUGGCACCGAGUCGGUGCUUUU |
| PAH1_hyb27 | 67 | UCAdCdAdGUUdCdGGGGGUAUACAGUUUUAGAGCUAGAAAUAGCAAGUUAAA |
| | | AUAAGGCUAGUCCGUUAUCAACUUGAAAAAGUGGCACCGAGUCGGUGCUUUU |
| PAH1_hyb28 | 68 | UCdAdCdAdGUUdCdGGGGGUAUACAGUUUUAGAGCUAGAAAUAGCAAGUUAA |
| | | AAUAAGGCUAGUCCGUUAUCAACUUGAAAAAGUGGCACCGAGUCGGUGCUUU |
| | | U |

Lightly modified guide oligonucleotides

| | | |
|---|---|---|
| Generic | 69 | mN*₃N₁₇GUUUUAGAGCUAGAAAUAGCAAGUUAAAAUAAGGCUAGUCCGUUAU |
| sequence | | CAACUUGAAAAAGUGGCACCGAGUCGGUGCU*mU*mU*mU |
| PAH1 | 70 | mU*mC*mA*CAGUUCGGGGGUAUACAGUUUUAGAGCUAGAAAUAGCAAGUUA |
| | | AAAUAAGGCUAGUCCGUUAUCAACUUGAAAAAGUGGCACCGAGUCGGUGCU* |
| | | mU*mU*mU |
| PAH2 | 71 | mC*mA*mC*AGUUCGGGGGUAUACAUGUUUUAGAGCUAGAAAUAGCAAGUUA |
| | | AAAUAAGGCUAGUCCGUUAUCAACUUGAAAAAGUGGCACCGAGUCGGUGCU* |
| | | mU*mU*mU |
| PAH1_hyb1 | 72 | mU*mC*dA*CAGUUCGGGGGUAUACAGUUUUAGAGCUAGAAAUAGCAAGUUA |
| | | AAAUAAGGCUAGUCCGUUAUCAACUUGAAAAAGUGGCACCGAGUCGGUGCU* |
| | | mU*mU*mU |
| PAH1_hyb2 | 73 | mU*mC*mA*dCAGUUCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb3 | 74 | mU*mC*mA*CdAGUUCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb4 | 75 | mU*mC*mA*CAdGUUCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb5 | 76 | mU*mC*mA*CAGdTUCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb6 | 77 | mU*mC*mA*CAGUdTCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb7 | 78 | mU*mC*mA*CAGUUdCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb8 | 79 | mU*mC*mA*CAGUUCdGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb9 | 80 | mU*mC*dA*dCAGUUCGGGGGUAUACAGUUUUAGAGCUAGAAAUAGCAAGUU |
| | | AAAAUAAGGCUAGUCCGUUAUCAACUUGAAAAAGUGGCACCGAGUCGGUGCU* |
| | | mU*mU*mU |
| PAH1_hyb10 | 81 | mU*mC*mA*dCdAGUUCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb11 | 82 | mU*mC*mA*CdAdGUUCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb12 | 83 | mU*mC*mA*CAdGdTUCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb13 | 84 | mU*mC*mA*CAGdTdTCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb14 | 85 | mU*mC*mA*CAGUdTdCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb15 | 86 | mU*mC*mA*CAGUUdCdGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb16 | 87 | mU*mC*dA*dCdAGUUCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb17 | 88 | mU*mC*mA*dCdAdGUUCGGGGGUAUACAGUUUUAGAGCUAGAAAUAGCAAG |
| | | UUAAAAUAAGGCUAGUCCGUUAUCAACUUGAAAAAGUGGCACCGAGUCGGUGC |
| | | U*mU*mU*mU |
| PAH1_hyb18 | 89 | mU*mC*mA*CdAdGdTUCGGGGGUAUACAGUUUUAGAGCUAGAAAUAGCAAG |
| | | UUAAAAUAAGGCUAGUCCGUUAUCAACUUGAAAAAGUGGCACCGAGUCGGUGC |
| | | U*mU*mU*mU |
| PAH1_hyb19 | 90 | mU*mC*mA*CAdGdTdTCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb20 | 91 | mU*mC*mA*CAGdTdTdCGGGGGUAUACAGUUUUAGAGCUAGAAAUAGCAAGU |
| | | UAAAAUAAGGCUAGUCCGUUAUCAACUUGAAAAAGUGGCACCGAGUCGGUGCU |
| | | *mU*mU*mU |
| PAH1_hyb21 | 92 | mU*mC*mA*CAGUdTdCdGGGGGUAUACAGUUUUAGAGCUAGAAAUAGCAAG |
| | | UUAAAAUAAGGCUAGUCCGUUAUCAACUUGAAAAAGUGGCACCGAGUCGGUGC |
| | | U*mU*mU*mU |
| PAH1_hyb22 | 93 | mU*mC*dA*dCdAdGUUCGGGGGUAUACAGUUUUAGAGCUAGAAAUAGCAAG |
| | | UUAAAAUAAGGCUAGUCCGUUAUCAACUUGAAAAAGUGGCACCGAGUCGGUGC |
| | | U*mU*mU*mU |
| PAH1_hyb23 | 94 | mU*mC*dA*dCAGUUdCdGGGGGUAUACAGUUUUAGAGCUAGAAAUAGCAAG |
| | | UUAAAAUAAGGCUAGUCCGUUAUCAACUUGAAAAAGUGGCACCGAGUCGGUGC |
| | | U*mU*mU*mU |
| PAH1_hyb24 | 95 | mU*mC*mA*dCdAGUUdCdGGGGGUAUACAGUUUUAGAGCUAGAAAUAGCAA |
| | | GUUAAAAUAAGGCUAGUCCGUUAUCAACUUGAAAAAGUGGCACCGAGUCGGU |
| | | GCU*mU*mU*mU |
| PAH1_hyb25 | 96 | mU*mC*mA*CdAdGUUdCdGGGGGUAUACAGUUUUAGAGCUAGAAAUAGCAA |
| | | GUUAAAAUAAGGCUAGUCCGUUAUCAACUUGAAAAAGUGGCACCGAGUCGGU |
| | | GCU*mU*mU*mU |
| PAH1_hyb26 | 97 | mU*mC*dA*dCdAGUUdCdGGGGGUAUACAGUUUUAGAGCUAGAAAUAGCAAG |
| | | UUAAAAUAAGGCUAGUCCGUUAUCAACUUGAAAAAGUGGCACCGAGUCGGUGC |
| | | U*mU*mU*mU |
| PAH1_hyb27 | 98 | mU*mC*mA*dCdAdGUUdCdGGGGGUAUACAGUUUUAGAGCUAGAAAUAGCA |
| | | AGUUAAAAUAAGGCUAGUCCGUUAUCAACUUGAAAAAGUGGCACCGAGUCGG |
| | | UGCU*mU*mU*mU |
| PAH1_hyb28 | 99 | mU*mC*dA*dCdAdGUUdCdGGGGGUAUACAGUUUUAGAGCUAGAAAUAGCAA |
| | | GUUAAAAUAAGGCUAGUCCGUUAUCAACUUGAAAAAGUGGCACCGAGUCGGU |
| | | GCU*mU*mU*mU |

Heavily modified guide oligonucleotides

| | | |
|---|---|---|
| Generic | 100 | mN*₃N₁₇GUUUUAGAmGmCmUmAmGmAmAmAmUmAmGmCAAGUUAAAAU |
| sequence | | AAGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| PAH1 | 101 | mU*mC*mA*CAGUUCGGGGGUAUACAGUUUUAGAmGmCmUmAmGmAmAm |
| | | AmUmAmGmCAAGUUAAAAUAAGGCUAGUCCGUUAUCAmAmCmUmUmGmA |
| | | mAmAmAmAmGmUmGmGmCmAmCmCmGmAmGmUmCmGmGmUmGmCm |
| | | U*mU*mU*mU |
| PAH2 | 102 | mC*mA*mC*AGUUCGGGGGUAUACAUGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb1 | 103 | mU*mC*dA*CAGUUCGGGGGUAUACAGUUUUAGAmGmCmUmAmGmAmAm |
| | | AmUmAmGmCAAGUUAAAAUAAGGCUAGUCCGUUAUCAmAmCmUmUmGmA |
| | | mAmAmAmAmGmUmGmGmCmAmCmCmGmAmGmUmCmGmGmUmGmCm |
| | | U*mU*mU*mU |
| PAH1_hyb2 | 104 | mU*mC*mA*dCAGUUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb3 | 105 | mU*mC*mA*CdAGUUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb4 | 106 | mU*mC*mA*CAdGUUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb5 | 107 | mU*mC*mA*CAGdTUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb6 | 108 | mU*mC*mA*CAGUdTCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb7 | 109 | mU*mC*mA*CAGUUdCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb8 | 110 | mU*mC*mA*CAGUUCdGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb9 | 111 | mU*mC*dA*dCAGUUCGGGGGUAUACAGUUUUAGAmGmCmUmAmGmAmAm |
| | | AmUmAmGmCAAGUUAAAAUAAGGCUAGUCCGUUAUCAmAmCmUmUmGmA |
| | | mAmAmAmAmGmUmGmGmCmAmCmCmGmAmGmUmCmGmGmUmGmCm |
| | | U*mU*mU*mU |
| PAH1_hyb10 | 112 | mU*mC*mA*dCdAGUUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb11 | 113 | mU*mC*mA*CdAdGUUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb12 | 114 | mU*mC*mA*CAdGdTUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb13 | 115 | mU*mC*mA*CAGdTdTCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb14 | 116 | mU*mC*mA*CAGUdTdCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb15 | 117 | mU*mC*mA*CAGUUdCdGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb16 | 118 | mU*mC*dA*dCdAGUUCGGGGGUAUACAGUUUUAGAmGmCmUmAmGmAmA |
| | | mAmUmAmGmCAAGUUAAAAUAAGGCUAGUCCGUUAUCAmAmCmUmUmGm |
| | | AmAmAmAmAmGmUmGmGmCmAmCmCmGmAmGmUmCmGmGmUmGmC |
| | | mU*mU*mU*mU |
| PAH1_hyb17 | 119 | mU*mC*mA*dCdAdGUUCGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb18 | 120 | mU*mC*mA*CdAdGdTUCGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb19 | 121 | mU*mC*mA*CAdGdTdTCGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb20 | 122 | mU*mC*mA*CAGdTdTdCGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb21 | 123 | mU*mC*mA*CAGUdTdCdGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb22 | 124 | mU*mC*dA*dCdAdGUUCGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb23 | 125 | mU*mC*dA*dCAGUUdCdGGGGGUAUACAGUUUUAGAmGmCmUmAmGmAm |
| | | AmAmUmAmGmCAAGUUAAAAUAAGGCUAGUCCGUUAUCAmAmCmUmUmG |
| | | mAmAmAmAmAmGmUmGmGmCmAmCmCmGmAmGmUmCmGmGmUmGm |
| | | CmU*mU*mU*mU |
| PAH1_hyb24 | 126 | mU*mC*mA*dCdAGUUdCdGGGGGUAUACAGUUUUAGAmGmCmUmAmGmA |
| | | mAmAmUmAmGmCAAGUUAAAAUAAGGCUAGUCCGUUAUCAmAmCmUmUm |
| | | GmAmAmAmAmAmGmUmGmGmCmAmCmCmGmAmGmUmCmGmGmUmG |
| | | mCmU*mU*mU*mU |
| PAH1_hyb25 | 127 | mU*mC*mA*CdAdGUUdCdGGGGGUAUACAGUUUUAGAmGmCmUmAmGmA |
| | | mAmAmUmAmGmCAAGUUAAAAUAAGGCUAGUCCGUUAUCAmAmCmUmUm |
| | | GmAmAmAmAmAmGmUmGmGmCmAmCmCmGmAmGmUmCmGmGmUmG |
| | | mCmU*mU*mU*mU |
| PAH1_hyb26 | 128 | mU*mC*dA*dCdAGUUdCdGGGGGUAUACAGUUUUAGAmGmCmUmAmGmA |
| | | mAmAmUmAmGmCAAGUUAAAAUAAGGCUAGUCCGUUAUCAmAmCmUmUm |
| | | GmAmAmAmAmAmGmUmGmGmCmAmCmCmGmAmGmUmCmGmGmUmG |
| | | mCmU*mU*mU*mU |
| PAH1_hyb27 | 129 | mU*mC*mA*dCdAdGUUdCdGGGGGUAUACAGUUUUAGAmGmCmUmAmGm |
| | | AmAmAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUm |
| | | GmCmU*mU*mU*mU |
| PAH1_hyb28 | 130 | mU*mC*dA*dCdAdGUUdCdGGGGGUAUACAGUUUUAGAmGmCmUmAmGmA |
| | | mAmAmUmAmGmCAAGUUAAAAUAAGGCUAGUCCGUUAUCAmAmCmUmUm |
| | | GmAmAmAmAmAmGmUmGmGmCmAmCmCmGmAmGmUmCmGmGmUmG |
| | | mCmU*mU*mU*mU |

m = 2′O-methyl group; * = 2′-ribo 3′-phosphorothioate linkage; d = deoxy (DNA) version of nucleotide

### Example 8

### ABE Activity in Mice Via LNP Delivery.

For LNP delivery, the PAH gRNAs with appropriately positioned 2′-O-methyl and phosphorothioate modifications were synthesized by Agilent. mRNA encoding the ABE was generated via in vitro transcription and purification by the University of Pennsylvania Engineered mRNA and Targeted Nanomedicine Core. The Core also formulated the mRNA with the gRNA into LNPs containing standard lipid components (ionizable cationic lipid, 1,2-distearoyl-sn-glycero-3-phosphocholine, cholesterol, and a PEG-lipid).

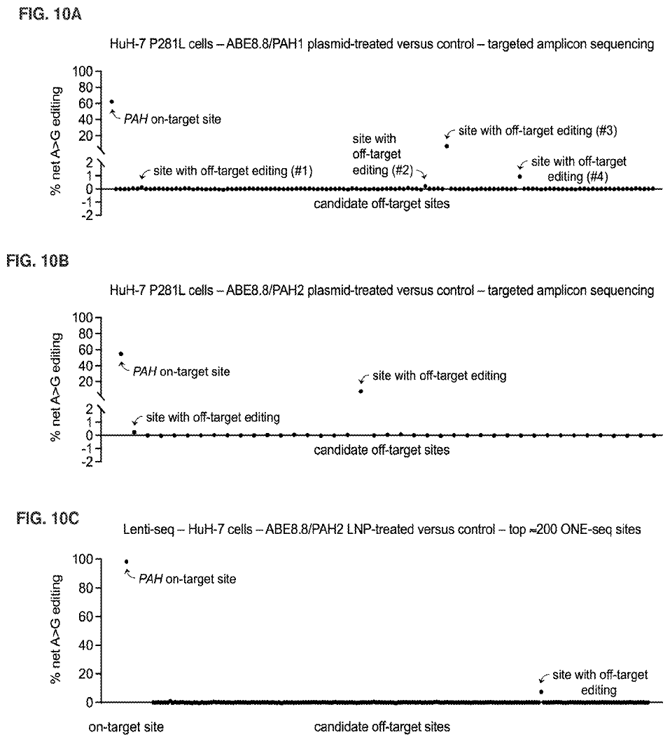

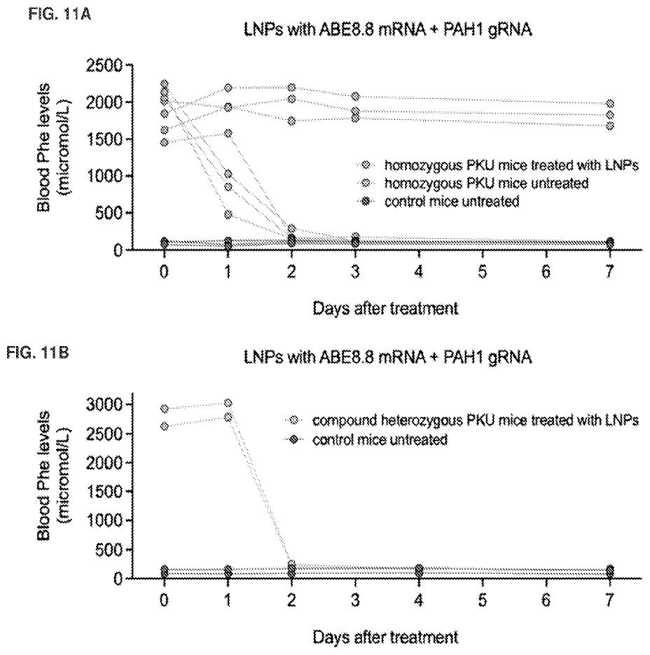

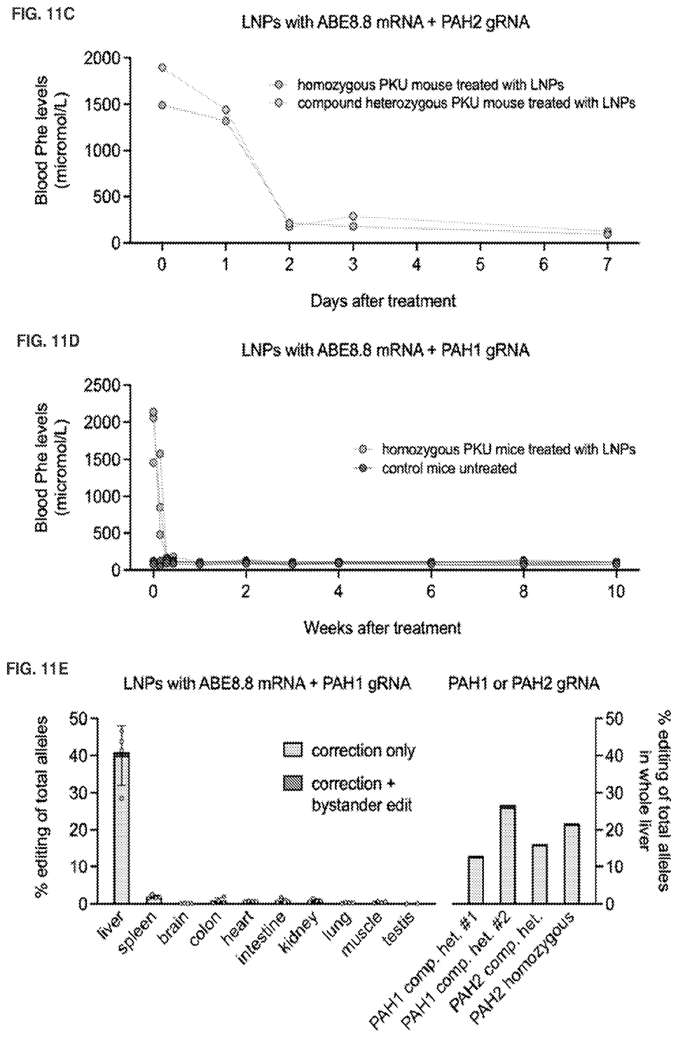

In a short-term study, four age-matched (8 weeks of age) homozygous P281L (PKU) mice were treated with ABE8.8/PAH1 LNPs, with three heterozygous P281L (non-PKU) colony mates and three untreated homozygous P281L (PKU) colony-mates serving as controls (FIG. 11A). At baseline, the PKU mice had blood phenylalanine (Phe) levels ranging from 1455-2242 mol/L, whereas the non-PKU mice had blood Phe levels <120 mol/L (similar to human profiles). Some of the treated mice displayed substantially decreased Phe levels at 24 hours after treatment (36% mean reduction for all treated mice). All the treated mice had largely normalized Phe levels at 48 hours after treatment (90% mean reduction) and were indistinguishable from non-PKU mice at 1 week after treatment (PKU, mean 104 mol/L; non-PKU, mean 96 mol/L).

Two additional short-term studies were undertaken. In one study, two 4-week-old compound heterozygous P281L (PKU) mice were treated with ABE8.8/PAH1 LNPs, with two heterozygous P281L (non-PKU) littermates serving as controls (FIG. 11B). In the other study, a 10-week-old homozygous P281L (PKU) mouse and a 10-week-old compound heterozygous P281L (PKU) mouse were treated with ABE8.8/PAH2 LNPs (FIG. 11C). In all cases, the treated PKU mice had largely normalized blood Phe levels by 48 hours after treatment (91% and 88% mean reductions for second and third short-term studies, respectively).

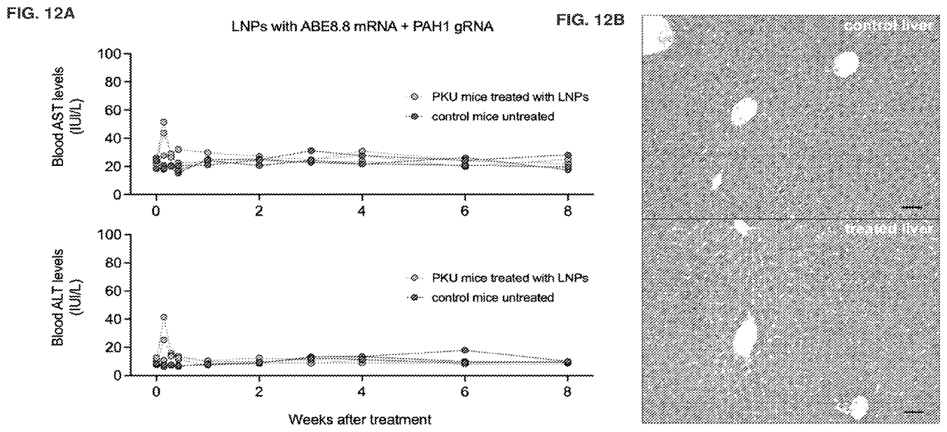

Three ABE8.8/PAH1 LNP-treated PKU mice and three control non-PKU mice from the first short-term study have been maintained in an ongoing long-term study; up to 10 weeks after treatment, the LNP-treated mice have maintained normal Phe levels (FIG. 11D). By 8 weeks after treatment, the hypopigmentation of the treated PKU mice had resolved (FIG. 7B). Aspartate aminotransferase (AST) and alanine aminotransferase (ALT) levels had slight rises in some mice at 24 hours after treatment, remaining within the normal ranges and resolving by 72 hours (FIG. 12A).

Four ABE8.8/PAH1 LNP-treated homozygous mice (including one from the original short-term study), two ABE8.8/PAH1 LNP-treated compound heterozygous mice (second short-term study), and two ABE8.8/PAH2 LNP-treated mice (third short-term study) were necropsied 1-2 weeks after treatment to assess editing in the liver and a variety of other organs. Corrective editing occurred predominantly in the liver, with low-level editing observed in the spleen and minimal editing in the other organs, consistent with prior LNP studies (FIG. 11E). The desired corrective editing in the liver with ABE8.8/PAH1 LNPs ranged from 28% to 47% in the homozygous mice and from 26% to 52% of the editable alleles in the compound heterozygous mouse (i.e., 13% to 26% of total alleles); with ABE8.8/PAH2 LNPs, 22% to 32% of the editable alleles (FIG. 11E). Very low levels of bystander editing were observed with PAH1 (mean 0.8%) and even less with PAH2 (mean 0.2%). Liver histology from the necropsied ABE8.8/PAH1 LNP-treated mouse from the first short-term study showed no evidence of pathology (FIG. 12B).

### REFERENCES

Anzalone A V, Koblan L W, Liu D R. Genome editing with CRISPR-Cas nucleases, base editors, transposases and prime editors. Nat Biotechnol. 2020 July; 38(7):824-844.
Anzalone A V, Randolph P B, Davis J R, Sousa A A, Koblan L W, Levy J M, Chen P J, Wilson C, Newby G A, Raguram A, Liu D R. Search-and-replace genome editing without double-strand breaks or donor DNA. Nature. 2019 December; 576(7785):149-157.
Banskota S, Raguram A, Suh S, Du S W, Davis J R, Choi E H, Wang X, Nielsen S C, Newby G A, Randolph P B, Osborn M J, Musunuru K, Palczewski K, Liu D R. Engineered virus-like particles for efficient in vivo delivery of therapeutic proteins. Cell. 2022 Jan. 20; 185(2):250-265.e16. Blau N, van Spronsen F J, Levy H L. Phenylketonuria. Lancet. 2010 Oct. 23; 376(9750):1417-27.
Cancellieri S, Zeng J, Lin L Y, Masillo F, Nguyen A, Bombieri N, Ciuculescu F, Katta V, Tsai S Q, Armant M, Giugno R, Bauer D E, Pinello L. Human genetic diversity modifies therapeutic gene editing off-target potential. bioRxiv. 2021 May 21.
Chadwick A C, Evitt N H, Lv W, Musunuru K. Reduced Blood Lipid Levels With In Vivo CRISPR-Cas9 Base Editing of ANGPTL3. Circulation. 2018 Feb. 27; 137(9):975-977.
Chadwick A C, Wang X, Musunuru K. In Vivo Base Editing of PCSK9 (Proprotein Convertase Subtilisin/Kexin Type 9) as a Therapeutic Alternative to Genome Editing. Arterioscler Thromb Vasc Biol. 2017 September; 37(9):1741-1747. doi: 10.1161/ATVBAHA.117.309881. Epub 2017 Jul. 27.
Chu S H, Packer M, Rees H, Lam D, Yu Y, Marshall J, Cheng L I, Lam D, Olins J, Ran F A, Liquori A, Gantzer B, Decker J, Born D, Barrera L, Hartigan A, Gaudelli N, Ciaramella G, Slaymaker I M. Rationally Designed Base Editors for Precise Editing of the Sickle Cell Disease Mutation. CRISPR J. 2021 April; 4(2):169-177.
Davis J R, Wang X, Witte I P, Huang T P, Levy J M, Raguram A, Banskota S, Seidah N G, Musunuru K, Liu D R. Efficient in vivo base editing via single adeno-associated viruses with size-optimized genomes encoding compact adenine base editors. Nat Biomed Eng. 2022 November; 6(11):1272-1283.
Donohoue P D, Pacesa M, Lau E, Vidal B, Irby M J, Nyer D B, Rotstein T, Banh L, Toh M S, Gibson J, Kohrs B, Baek K, Owen ALG, Slorach E M, van Overbeek M, Fuller C K, May A P, Jinek M, Cameron P. Conformational control of Cas9 by CRISPR hybrid RNA-DNA guides mitigates off-target activity in T cells. Mol Cell. 2021 Sep. 2; 81(17):3637-3649.e5.
Essalmani R, Weider E, Marcinkiewicz J, Chamberland A, Susan-Resiga D, Roubtsova A, Seidah N G, Prat A. A single domain antibody against the Cys- and His-rich domain of PCSK9 and evolocumab exhibit different inhibition mechanisms in humanized PCSK9 mice. Biol Chem. 2018 Nov. 27; 399(12):1363-1374.
Gaudelli N M, Komor A C, Rees H A, Packer M S, Badran A H, Bryson D I, Liu D R. Programmable base editing of A⋅T to G⋅C in genomic DNA without DNA cleavage. Nature. 2017 Nov. 23; 551(7681):464-471.
Gaudelli N M, Lam D K, Rees H A, Solá-Esteves N M, Barrera L A, Born D A, Edwards A, Gehrke J M, Lee S J, Liquori A J, Murray R, Packer M S, Rinaldi C, Slaymaker I M, Yen J, Young L E, Ciaramella G. Directed evolution of adenine base editors with increased activity and therapeutic application. Nat Biotechnol. 2020 July; 38(7):892-900.
Gillmore J D, Gane E, Taubel J, Kao J, Fontana M, Maitland M L, Seitzer J, O'Connell D, Walsh K R, Wood K, Phillips J, Xu Y, Amaral A, Boyd A P, Cehelsky J E, McKee M D, Schiermeier A, Harari O, Murphy A, Kyratsous C A, Zambrowicz B, Soltys R, Gutstein D E, Leonard J, Sepp-Lorenzino L, Lebwohl D. CRISPR-Cas9 In Vivo Gene Editing for Transthyretin Amyloidosis. N Engl J Med. 2021 Aug. 5; 385(6):493-502.
Grisch-Chan H M, Schwank G, Harding C O, Thöny B. State-of-the-Art 2019 on Gene Therapy for Phenylketonuria. Hum Gene Ther. 2019 October; 30(10):1274-1283.
Hillert A, Anikster Y, Belanger-Quintana A, Burlina A, Burton B K, Carducci C, Chiesa A E, Christodoulou J, Ðordević M, Desviat L R, Eliyahu A, Evers R A F, Fajkusova L, Feillet F, Bonfim-Freitas P E, Giżewska M, Gundorova P, Karall D, Kneller K, Kutsev S I, Leuzzi V, Levy H L, Lichter-Konecki U, Muntau A C, Namour F, Oltarzewski M, Paras A, Perez B, Polak E, Polyakov A V, Porta F, Rohrbach M, Scholl-Bürgi S, Spécola N, Stojiljković M, Shen N, Santana-da Silva L C, Skouma A, van Spronsen F, Stoppioni V, Thöny B, Trefz F K, Vockley J, Yu Y, Zschocke J, Hoffmann G F, Garbade S F, Blau N. The Genetic Landscape and Epidemiology of Phenylketonuria. Am J Hum Genet. 2020 Aug. 6; 107(2):234-250.
Jinek M, Chylinski K, Fonfara I, Hauer M, Doudna J A, Charpentier E. A programmable dual-RNA-guided DNA endonuclease in adaptive bacterial immunity. Science. 2012 Aug. 17; 337(6096):816-21.
Kasiewicz L N, Biswas S, Beach A, Ren H, Dutta C, Mazzola A M, Rohde E, Chadwick A, Cheng C, Musunuru K, Kathiresan S, Malyala P, Rajeev K G, Bellinger A M. Lipid nanoparticles incorporating a GalNAc ligand enable in vivo liver ANGPTL3 editing in wild-type and somatic LDLR knockout non-human primates. bioRxiv. 2021 Nov. 8.
Kim D, Kim D E, Lee G, Cho S I, Kim J S. Genome-wide target specificity of CRISPR RNA-guided adenine base editors. Nat Biotechnol. 2019 April; 37(4):430-435.
Kingwell K. Base editors hit the clinic. Nat. Rev. Drug Discov. 21, 545-547 (2022).
Komor A C, Kim Y B, Packer M S, Zuris J A, Liu D R. Programmable editing of a target base in genomic DNA without double-stranded DNA cleavage. Nature. 2016 May 19; 533(7603):420-4.
Leuders S, Wolfgart E, Ott T, du Moulin M, van Teeffelen-Heithoff A, Vogelpohl L, Och U, Marquardt T, Weglage J, Feldmann R, Rutsch F. Influence of PAH Genotype on Sapropterin Response in PKU: Results of a Single-Center Cohort Study. JEVID Rep. 2014; 13:101-9.
Levy H L, Sarkissian C N, Scriver C R. Phenylalanine ammonia lyase (PAL): From discovery to enzyme substitution therapy for phenylketonuria. Mol Genet Metab. 2018 August; 124(4):223-229. Levy J M, Yeh W H, Pendse N, Davis J R, Hennessey E, Butcher R, Koblan L W, Comander J, Liu Q, Liu D R. Cytosine and adenine base editing of the brain, liver, retina, heart and skeletal muscle of mice via adeno-associated viruses. Nat Biomed Eng. 2020 January; 4(1):97-110.
Liang P, Xie X, Zhi S, Sun H, Zhang X, Chen Y, Chen Y, Xiong Y, Ma W, Liu D, Huang J, Songyang Z. Genome-wide profiling of adenine base editor specificity by EndoV-seq. Nat Commun. 2019 Jan. 8; 10(1):67.
Musunuru K, Chadwick A C, Mizoguchi T, Garcia S P, DeNizio J E, Reiss C W, Wang K, Iyer S, Dutta C, Clendaniel V, Amaonye M, Beach A, Berth K, Biswas S, Braun M C, Chen H M, Colace T V, Ganey J D, Gangopadhyay S A, Garrity R, Kasiewicz L N, Lavoie J, Madsen J A, Matsumoto Y, Mazzola A M, Nasrullah Y S, Nneji J, Ren H, Sanjeev A, Shay M, Stahley M R, Fan S H Y, Tam Y K, Gaudelli N M, Ciaramella G, Stolz L E, Malyala P, Cheng C J, Rajeev K G, Rohde E, Bellinger A M, Kathiresan S. In vivo CRISPR base editing of PCSK9 durably lowers cholesterol in primates. Nature. 2021 May; 593(7859):429-434.
Petri K, Kim D Y, Sasaki K E, Canver M C, Wang X, Shah H, Lee H, Horng J E, Clement K, Iyer S, Garcia S P, Guo J A, Newby G A, Pinello L, Liu D R, Aryee M J, Musunuru K, Joung J K, Pattanayak V. Global-scale CRISPR gene editor specificity profiling by ONE-seq identifies population-specific, variant off-target effects. bioRxiv. 2021 Apr. 5.
Regier D S, Greene C L. Phenylalanine Hydroxylase Deficiency. 2017 Jan. 5. In: Adam M P, Ardinger H H, Pagon R A, Wallace S E, Bean L J H, Gripp K W, Mirzaa G M, Amemiya A, editors. GeneReviews® [Internet]. Seattle (W A): University of Washington, Seattle; 1993-2022.
Richter M F, Zhao K T, Eton E, Lapinaite A, Newby G A, Thuronyi B W, Wilson C, Koblan L W, Zeng J, Bauer D E, Doudna J A, Liu D R. Phage-assisted evolution of an adenine base editor with improved Cas domain compatibility and activity. Nat Biotechnol. 2020 July; 38(7):883-891.
Rossidis A C, Stratigis J D, Chadwick A C, Hartman H A, Ahn N J, Li H, Singh K, Coons B E, Li L, Lv W, Zoltick P W, Alapati D, Zacharias W, Jain R, Morrisey E E, Musunuru K, Peranteau W H. In utero CRISPR-mediated therapeutic editing of metabolic genes. Nat Med. 2018 October; 24(10):1513-1518.
Rothgangl T, Dennis M K, Lin P J C, Oka R, Witzigmann D, Villiger L, Qi W, Hruzova M, Kissling L, Lenggenhager D, Borrelli C, Egli S, Frey N, Bakker N, Walker J A 2nd, Kadina A P, Victorov D V, Pacesa M, Kreutzer S, Kontarakis Z, Moor A, Jinek M, Weissman D, Stoffel M, van Boxtel R, Holden K, Pardi N, Thöny B, Häberle J, Tam Y K, Semple S C, Schwank G. In vivo adenine base editing of PCSK9 in macaques reduces LDL cholesterol levels. Nat Biotechnol. 2021 August; 39(8):949-957.
Strecker J, Jones S, Koopal B, Schmid-Burgk J, Zetsche B, Gao L, Makarova K S, Koonin E V, Zhang F. Engineering of CRISPR-Cas12b for human genome editing. Nat Commun. 2019 Jan. 22; 10(1):212. doi: 10.1038/s41467-018-08224-4.
van Spronsen F J, Blau N, Harding C, Burlina A, Longo N, Bosch A M. Phenylketonuria. Nat Rev Dis Primers. 2021 May 20; 7(1):36.
Villiger L, Grisch-Chan H M, Lindsay H, Ringnalda F, Pogliano C B, Allegri G, Fingerhut R, Haberle J, Matos J, Robinson M D, Thöny B, Schwank G. Treatment of a metabolic liver disease by in vivo genome base editing in adult mice. Nat Med. 2018 October; 24(10):1519-1525.
Wang L, Breton C, Warzecha C C, Bell P, Yan H, He Z, White J, Zhu Y, Li M, Buza E L, Jantz D, Wilson J M. Long-term stable reduction of low-density lipoprotein in nonhuman primates following in vivo genome editing of PCSK9. Mol Ther. 2021 Jun. 2; 29(6):2019-2029.
Zetsche B, Gootenberg J S, Abudayyeh 00, Slaymaker I M, Makarova K S, Essletzbichler P, Volz S E, Joung J, van der Oost J, Regev A, Koonin E V, Zhang F. Cpf1 is a single RNA-guided endonuclease of a class 2 CRISPR-Cas system. Cell. 2015 Oct. 22; 163(3):759-71.
Zheng Z, Liebers M, Zhelyazkova B, Cao Y, Panditi D, Lynch K D, Chen J, Robinson H E, Shim H S, Chmielecki J, Pao W, Engelman J A, Iafrate A J, Le L P. Anchored multiplex PCR for targeted next-generation sequencing. Nat Med. 2014 December; 20(12):1479-84.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for editing a phenylalanine hydroxylase (PAH) encoding polynucleotide comprising mutation associated with phenylketonuria (PKU), the method comprising contacting the PAH polynucleotide with a base editor in complex with at least one guide polynucleotides, wherein the base editor comprises a polynucleotide programmable DNA binding domain and an adenosine deaminase domain, and wherein one or more of said guide polynucleotides target said base editor to effect an A⋅T to G⋅C alteration of the mutation associated with PKU.

2. The method of claim 1, wherein the contacting is in a cell in vivo or in vitro, and said cell is a eukaryotic cell, a mammalian cell, or human cell.

3.-4. (canceled)

5. The method of claim 1, wherein the mutation is one or more of c. 842C>T (p.Pro281Leu), c.1222C>T (p.Arg408Trp), c.1066-11G>A, c.782G>A (p.Arg261Gln), c.728G>A (p.Arg243Gln), c.1315+1G>A, and c.473G>A (p.Arg158Gln).

6. The method of claim 5, wherein the polynucleotide programmable DNA binding domain is a Streptococcus pyogenes Cas9 (SpCas9) or Staphylococcus aureus Cas9 (SaCas9) or a variant thereof or wherein the polynucleotide programmable DNA binding domain comprises a modified SpCas9 having an altered protospacer-adjacent motif (PAM) specificity.

7. (canceled)

8. The method of claim 6, wherein the polynucleotide programmable DNA binding domain is a nuclease inactive or nickase variant and the adenosine deaminase domain is capable of deaminating adenosine in deoxyribonucleic acid (DNA).

9. (canceled)

10. The method of claim 8, wherein the adenosine deaminase is a TadA deaminase or a variant thereof.

11. The method of claim 5, wherein the base editor is in complex with a single guide RNA (sgRNA) comprising a nucleic acid sequence complementary to a nucleic acid sequence comprising the mutation associated with PKU.

12. A cell produced by introducing into the cell, or a progenitor thereof:
a) a base editor, or a polynucleotide encoding said base editor, to said cell, wherein said base editor comprises a polynucleotide programmable DNA binding domain and an adenosine deaminase domain; and
b) one or more guide polynucleotides that target the base editor to effect an A⋅T to G⋅C alteration of the mutation associated with PKU.

13. The cell of claim 12, wherein the cell is a hepatocyte expressing a PAH polypeptide.

14. (canceled)

15. The cell of claim 12, wherein the cell is from a subject having PKU, wherein the base editor is in complex with a single guide RNA (sgRNA) comprising a nucleic acid sequence complementary to a PAH encoding nucleic acid sequence comprising the mutation associated with PKU.

16. The cell of claim 12, wherein the polynucleotide programmable DNA binding domain is a Streptococcus pyogenes Cas9 (SpCas9) or variant thereof, or the polynucleotide programmable DNA binding domain comprises a modified SpCas9 having an altered protospacer-adjacent motif (PAM) specificity.

17. (canceled)

18. The cell of claim 16, wherein the modified SpCas9 has specificity for the nucleic acid sequence 5′-NGG-3′.

19. The cell of any one of claim 12, wherein the polynucleotide programmable DNA binding domain is a nuclease inactive or nickase variant and the adenosine deaminase domain is capable of deaminating adenosine in deoxyribonucleic acid (DNA).

20.-21. (canceled)

22. The cell of claim 12 comprising an adenosine base editor/guide polynucleotide set which corrects a mutation causing PKU comprising:
(i) a modified SpCas9 or SaCas9;
(ii) an adenosine deaminase or functional fragment thereof; and
iii) a guide polynucleotide that targets the base editor to effect an A⋅T to G⋅C alteration of the mutation associated with PKU, wherein the base editor/guide polynucleotide set of claim 22, wherein said mutation is PAH c.842C>T (p.Pro281Leu) and said guide polynucleotide has a sequence of SEQ ID NO: 1, SEQ ID NO: 3, SEQ ID NO: 101, or SEQ ID NO:102 or is a hybrid gRNA having a sequence listed in Table 5 or wherein said guide polynucleotide comprises a nucleic acid sequence complementary to a PAH encoding nucleic acid sequence of SEQ ID NO:5 or SEQ ID NO:6.

23.-25. (canceled)

26. A method of treating PKU in a subject comprising administering to said subject an effective amount the adenosine base editor/guide polynucleotide set claim 22, wherein the subject is a mammal or a human.

27. (canceled)

28. The method of claim 26, comprising delivering the base editor, or polynucleotide encoding said base editor, and said one or more guide polynucleotides to a liver cell of the subject.

29. (canceled)

30. The method of claim 28, wherein said base editor/guide polynucleotide set are encapsulated in a lipid nanoparticle formulation and delivered to the liver of said subject, said formulation comprises ionizable cationic lipid, 1.2-distearoyl-sn-glycero-3-phosphocholine, cholesterol, and a PEG-lipid.

31. (canceled)

32. The method of claim 28, wherein said base editor and guide polynucleotide are delivered to hepatocytes in a single or dual AAV vector system or in a virus-like particle.

33. (canceled)

34. A transgenic mouse comprising a humanized Pah gene comprising a mutation associated with PKU selected from one or more of c. 842C>T (p.Pro281Leu), c.1222C>T (p.Arg408Trp), c.1066-11G>A, c.782G>A (p.Arg261Gln), c.728G>A (p.Arg243Gln), c.1315+1G>A, and c.473G>A (p.Arg15Gln).

35. (canceled)

36. The transgenic mouse of claim 34, wherein said mutation is PAH c.842C>T (P.Pro281Leu).


SEQUENCE LISTING (130 sequences)

SEQ ID NO: 1 - RNA, 100 nt, synthetic construct
  source 1..100: mol_type = other RNA
tcacagttcgggggtatacagttttagagctagaaatagcaagttaaaataaggctagtccgttatcaacttgaaaaagtggcaccgagtcggtgctttt

SEQ ID NO: 2 - RNA, 20 nt, synthetic construct
  source 1..20: mol_type = other RNA
tcacagttcgggggtataca

SEQ ID NO: 3 - RNA, 100 nt, synthetic construct
  source 1..100: mol_type = other RNA
cacagttcgggggtatacatgttttagagctagaaatagcaagttaaaataaggctagtccgttatcaacttgaaaaagtggcaccgagtcggtgctttt

SEQ ID NO: 4 - RNA, 20 nt, synthetic construct
  source 1..20: mol_type = other RNA
cacagttcgggggtatacat

SEQ ID NO: 5 - DNA, 20 bp, synthetic construct
  misc_feature 1..20: note = PAH encoding nucleic acid sequence
  source 1..20: mol_type = other DNA
tgtatacccccgaactgtga

SEQ ID NO: 6 - DNA, 20 bp, synthetic construct
  misc_feature 1..20: note = PAH encoding nucleic acid sequence
  source 1..20: mol_type = other DNA
atgtatacccccgaactgtg

SEQ ID NO: 7 - protein, 1588 aa, synthetic construct
  REGION 1..1588: note = ABE8.8-m
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 8 - DNA, 39 bp, synthetic construct
  misc_feature 1..39: note = the site of the PAH
  source 1..39: mol_type = other DNA
gacagtactcacggttcgggggtatacatgggcttggat

SEQ ID NO: 9 - DNA, 23 bp, synthetic construct
  misc_feature 1..23: note = candidate sites for DNA editing
  source 1..23: mol_type = other DNA
tcacagttcgggggtatacatgg

SEQ ID NO: 10 - DNA, 23 bp, synthetic construct
  misc_feature 1..23: note = candidate sites for DNA editing
  source 1..23: mol_type = other DNA
gtgccaagcccccatatgtaggg

SEQ ID NO: 11 - DNA, 23 bp, synthetic construct
  misc_feature 1..23: note = candidate sites for DNA editing
  source 1..23: mol_type = other DNA
cccgcaccttggcgcagcggtgg

SEQ ID NO: 12 - DNA, 20 bp, synthetic construct
  source 1..20: mol_type = other DNA
  misc_feature 1..2: note = RNA
  misc_feature 3: note = DNA
  misc_feature 4..20: note = RNA
tcacagttcgggggtataca

SEQ ID NO: 13 - DNA, 20 bp, synthetic construct
  source 1..20: mol_type = other DNA
  misc_feature 1..3: note = RNA
  misc_feature 4: note = DNA
  misc_feature 5..20: note = RNA
tcacagttcgggggtataca

SEQ ID NO: 14 - DNA, 20 bp, synthetic construct
  source 1..20: mol_type = other DNA
  misc_feature 1..4: note = RNA
  misc_feature 5: note = DNA
  misc_feature 6..20: note = RNA
tcacagttcgggggtataca

SEQ ID NO: 15 - DNA, 20 bp, synthetic construct
  source 1..20: mol_type = other DNA
  misc_feature 1..5: note = RNA
  misc_feature 6: note = DNA
  misc_feature 7..20: note = RNA
tcacagttcgggggtataca

SEQ ID NO: 16 - DNA, 20 bp, synthetic construct
  source 1..20: mol_type = other DNA
  misc_feature 1..6: note = RNA
  misc_feature 7: note = DNA
  misc_feature 8..20: note = RNA
tcacagttcgggggtataca

SEQ ID NO: 17 - DNA, 20 bp, synthetic construct
  source 1..20: mol_type = other DNA
  misc_feature 1..7: note = RNA
  misc_feature 8: note = DNA
  misc_feature 9..20: note = RNA
tcacagttcgggggtataca

SEQ ID NO: 18 - DNA, 20 bp, synthetic construct
  source 1..20: mol_type = other DNA
  misc_feature 1..8: note = RNA
  misc_feature 9: note = DNA
  misc_feature 10..20: note = RNA
tcacagttcgggggtataca

SEQ ID NO: 19 - DNA, 20 bp, synthetic construct
  source 1..20: mol_type = other DNA
  misc_feature 1..9: note = RNA
  misc_feature 10: note = DNA
  misc_feature 11..20: note = RNA
tcacagttcgggggtataca

SEQ ID NO: 20 - DNA, 20 bp, synthetic construct
  source 1..20: mol_type = other DNA
  misc_feature 1..2: note = RNA
  misc_feature 3..4: note = DNA
  misc_feature 5..20: note = RNA
tcacagttcgggggtataca

SEQ ID NO: 21 - DNA, 20 bp, synthetic construct
  source 1..20: mol_type = other DNA
  misc_feature 1..3: note = RNA
  misc_feature 4..5: note = DNA
  misc_feature 6..20: note = RNA
tcacagttcgggggtataca

SEQ ID NO: 22 - DNA, 20 bp, synthetic construct
  source 1..20: mol_type = other DNA
  misc_feature 1..4: note = RNA
  misc_feature 5..6: note = DNA
  misc_feature 7..20: note = RNA
tcacagttcgggggtataca

SEQ ID NO: 23 - DNA, 20 bp, synthetic construct
  source 1..20: mol_type = other DNA
  misc_feature 1..5: note = RNA
  misc_feature 6..7: note = DNA
  misc_feature 8..20: note = RNA
tcacagttcgggggtataca

SEQ ID NO: 24 - DNA, 20 bp, synthetic construct
  source 1..20: mol_type = other DNA
  misc_feature 1..6: note = RNA
  misc_feature 7..8: note = DNA
  misc_feature 9..20: note = RNA
tcacagttcgggggtataca

SEQ ID NO: 25 - DNA, 20 bp, synthetic construct
  source 1..20: mol_type = other DNA
  misc_feature 1..7: note = RNA
  misc_feature 8..9: note = DNA
  misc_feature 10..20: note = RNA
tcacagttcgggggtataca

SEQ ID NO: 26 - DNA, 20 bp, synthetic construct
  source 1..20: mol_type = other DNA
  misc_feature 1..8: note = RNA
  misc_feature 9..10: note = DNA
  misc_feature 11..20: note = RNA
tcacagttcgggggtataca

SEQ ID NO: 27 - DNA, 20 bp, synthetic construct
  source 1..20: mol_type = other DNA
  misc_feature 1..2: note = RNA
  misc_feature 3..5: note = DNA
  misc_feature 6..20: note = RNA
tcacagttcgggggtataca

SEQ ID NO: 28 - DNA, 20 bp, synthetic construct
  source 1..20: mol_type = other DNA
  misc_feature 1..3: note = RNA
  misc_feature 4..6: note = DNA
  misc_feature 7..20: note = RNA
tcacagttcgggggtataca

SEQ ID NO: 29 - DNA, 20 bp, synthetic construct
  source 1..20: mol_type = other DNA
  misc_feature 1..4: note = RNA
  misc_feature 5..7: note = DNA
  misc_feature 8..20: note = RNA
tcacagttcgggggtataca

SEQ ID NO: 30 - DNA, 20 bp, synthetic construct
  source 1..20: mol_type = other DNA
  misc_feature 1..5: note = RNA
  misc_feature 6..8: note = DNA
  misc_feature 9..20: note = RNA
tcacagttcgggggtataca

SEQ ID NO: 31 - DNA, 20 bp, synthetic construct
  source 1..20: mol_type = other DNA
  misc_feature 1..6: note = RNA
  misc_feature 7..9: note = DNA
  misc_feature 10..20: note = RNA
tcacagttcgggggtataca

SEQ ID NO: 32 - DNA, 20 bp, synthetic construct
  source 1..20: mol_type = other DNA
  misc_feature 1..8: note = RNA
  misc_feature 9..10: note = DNA
  misc_feature 11..20: note = RNA
tcacagttcgggggtataca

SEQ ID NO: 33 - DNA, 20 bp, synthetic construct
  source 1..20: mol_type = other DNA
  misc_feature 1..2: note = RNA
  misc_feature 3..6: note = DNA
  misc_feature 7..20: note = RNA
tcacagttcgggggtataca

SEQ ID NO: 34 - DNA, 20 bp, synthetic construct
  source 1..20: mol_type = other DNA
  misc_feature 1..2: note = RNA
  misc_feature 3..4: note = DNA
  misc_feature 5..8: note = RNA
  misc_feature 9..10: note = DNA
  misc_feature 11..20: note = RNA
tcacagttcgggggtataca

SEQ ID NO: 35 - DNA, 20 bp, synthetic construct
  misc_feature 1..3: note = RNA
  misc_feature 4..5: note = DNA
  misc_feature 6..8: note = RNA
  misc_feature 9..10: note = DNA
  misc_feature 11..20: note = RNA
  source 1..20: mol_type = other DNA
tcacagttcgggggtataca

SEQ ID NO: 36 - DNA, 20 bp, synthetic construct
  misc_feature 1..4: note = RNA
  misc_feature 5..6: note = DNA
  misc_feature 7..8: note = RNA
  misc_feature 9..10: note = DNA
  misc_feature 11..20: note = RNA
  source 1..20: mol_type = other DNA
tcacagttcgggggtataca

SEQ ID NO: 37 - DNA, 20 bp, synthetic construct
  misc_feature 1..2: note = RNA
  misc_feature 3..5: note = DNA
  misc_feature 6..8: note = RNA
  misc_feature 9..10: note = DNA
  misc_feature 11..20: note = RNA
  source 1..20: mol_type = other DNA
tcacagttcgggggtataca

SEQ ID NO: 38 - DNA, 20 bp, synthetic construct
  misc_feature 1..3: note = RNA
  misc_feature 4..6: note = DNA
  misc_feature 7..8: note = RNA
  misc_feature 9..10: note = DNA
  misc_feature 11..20: note = RNA
  source 1..20: mol_type = other DNA
tcacagttcgggggtataca

SEQ ID NO: 39 - DNA, 20 bp, synthetic construct
  misc_feature 1..2: note = RNA
  misc_feature 3..6: note = DNA
  misc_feature 7..8: note = RNA
  misc_feature 9..10: note = DNA
  misc_feature 11..20: note = RNA
  source 1..20: mol_type = other DNA
tcacagttcgggggtataca

SEQ ID NO: 40 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 41 - DNA, 100 bp, synthetic construct
  misc_feature 1..2: note = RNA
  misc_feature 3: note = DNA
  misc_feature 4..100: note = RNA
  source 1..100: mol_type = other DNA
tcacagttcgggggtatacagttttagagctagaaatagcaagttaaaataaggctagtccgttatcaacttgaaaaagtggcaccgagtcggtgctttt

SEQ ID NO: 42 - DNA, 100 bp, synthetic construct; identical to SEQ ID NO: 41
  misc_feature 1..3: note = RNA
  misc_feature 4: note = DNA
  misc_feature 5..100: note = RNA
  source 1..100: mol_type = other DNA

SEQ ID NO: 43 - DNA, 100 bp, synthetic construct; identical to SEQ ID NO: 41
  misc_feature 1..4: note = RNA
  misc_feature 5: note = DNA
  misc_feature 6..100: note = RNA
  source 1..100: mol_type = other DNA

SEQ ID NO: 44 - DNA, 100 bp, synthetic construct; identical to SEQ ID NO: 41
  misc_feature 1..5: note = RNA
  misc_feature 6: note = DNA
  misc_feature 7..100: note = RNA
  source 1..100: mol_type = other DNA

SEQ ID NO: 45 - DNA, 100 bp, synthetic construct; identical to SEQ ID NO: 41
  misc_feature 1..6: note = RNA
  misc_feature 7: note = DNA
  misc_feature 8..100: note = RNA
  source 1..100: mol_type = other DNA

SEQ ID NO: 46 - DNA, 100 bp, synthetic construct; identical to SEQ ID NO: 41
  misc_feature 1..7: note = RNA
  misc_feature 8: note = DNA
  misc_feature 9..100: note = RNA
  source 1..100: mol_type = other DNA

SEQ ID NO: 47 - DNA, 100 bp, synthetic construct; identical to SEQ ID NO: 41
  misc_feature 1..8: note = RNA
  misc_feature 9: note = DNA
  misc_feature 10..100: note = RNA
  source 1..100: mol_type = other DNA

SEQ ID NO: 48 - DNA, 100 bp, synthetic construct; identical to SEQ ID NO: 41
  misc_feature 1..9: note = RNA
  misc_feature 10: note = DNA
  misc_feature 11..100: note = RNA
  source 1..100: mol_type = other DNA

SEQ ID NO: 49 - DNA, 100 bp, synthetic construct; identical to SEQ ID NO: 41
  misc_feature 1..2: note = RNA
  misc_feature 3..4: note = DNA
  misc_feature 5..100: note = RNA
  source 1..100: mol_type = other DNA

SEQ ID NO: 50 - DNA, 100 bp, synthetic construct; identical to SEQ ID NO: 41
  misc_feature 1..3: note = RNA
  misc_feature 4..5: note = DNA
  misc_feature 6..100: note = RNA
  source 1..100: mol_type = other DNA

SEQ ID NO: 51 - DNA, 100 bp, synthetic construct; identical to SEQ ID NO: 41
  misc_feature 1..4: note = RNA
  misc_feature 5..6: note = DNA
  misc_feature 7..100: note = RNA
  source 1..100: mol_type = other DNA

SEQ ID NO: 52 - DNA, 100 bp, synthetic construct; identical to SEQ ID NO: 41
  misc_feature 1..5: note = RNA
  misc_feature 6..7: note = DNA
  misc_feature 8..100: note = RNA
  source 1..100: mol_type = other DNA

SEQ ID NO: 53 - DNA, 100 bp, synthetic construct; identical to SEQ ID NO: 41
  misc_feature 1..6: note = RNA
  misc_feature 7..8: note = DNA
  misc_feature 9..100: note = RNA
  source 1..100: mol_type = other DNA

SEQ ID NO: 54 - DNA, 100 bp, synthetic construct; identical to SEQ ID NO: 41
  misc_feature 1..7: note = RNA
  misc_feature 8..9: note = DNA
  misc_feature 10..100: note = RNA
  source 1..100: mol_type = other DNA

SEQ ID NO: 55 - DNA, 100 bp, synthetic construct; identical to SEQ ID NO: 41
  misc_feature 1..8: note = RNA
  misc_feature 9..10: note = DNA
  misc_feature 11..100: note = RNA
  source 1..100: mol_type = other DNA

SEQ ID NO: 56 - DNA, 100 bp, synthetic construct; identical to SEQ ID NO: 41
  misc_feature 1..2: note = RNA
  misc_feature 3..5: note = DNA
  misc_feature 6..100: note = RNA
  source 1..100: mol_type = other DNA

SEQ ID NO: 57 - DNA, 100 bp, synthetic construct; identical to SEQ ID NO: 41
  misc_feature 1..3: note = RNA
  misc_feature 4..6: note = DNA
  misc_feature 7..100: note = RNA
  source 1..100: mol_type = other DNA

SEQ ID NO: 58 - DNA, 100 bp, synthetic construct; identical to SEQ ID NO: 41
  misc_feature 1..4: note = RNA
  misc_feature 5..7: note = DNA
  misc_feature 8..100: note = RNA
  source 1..100: mol_type = other DNA

SEQ ID NO: 59 - DNA, 100 bp, synthetic construct; identical to SEQ ID NO: 41
  misc_feature 1..5: note = RNA
  misc_feature 6..8: note = DNA
  misc_feature 9..100: note = RNA
  source 1..100: mol_type = other DNA

SEQ ID NO: 60 - DNA, 100 bp, synthetic construct; identical to SEQ ID NO: 41
  misc_feature 1..6: note = RNA
  misc_feature 7..9: note = DNA
  misc_feature 10..100: note = RNA
  source 1..100: mol_type = other DNA

SEQ ID NO: 61 - DNA, 100 bp, synthetic construct; identical to SEQ ID NO: 41
  misc_feature 1..7: note = RNA
  misc_feature 8..10: note = DNA
  misc_feature 11..100: note = RNA
  source 1..100: mol_type = other DNA

SEQ ID NO: 62 - DNA, 100 bp, synthetic construct; identical to SEQ ID NO: 41
  misc_feature 1..2: note = RNA
  misc_feature 3..6: note = DNA
  misc_feature 7..100: note = RNA
  source 1..100: mol_type = other DNA

SEQ ID NO: 63 - DNA, 100 bp, synthetic construct; identical to SEQ ID NO: 41
  misc_feature 1..2: note = RNA
  misc_feature 3..4: note = DNA
  misc_feature 5..8: note = RNA
  misc_feature 9..10: note = DNA
  misc_feature 11..100: note = RNA
  source 1..100: mol_type = other DNA

SEQ ID NO: 64 - DNA, 100 bp, synthetic construct; identical to SEQ ID NO: 41
  misc_feature 1..3: note = RNA
  misc_feature 4..5: note = DNA
  misc_feature 6..8: note = RNA
  misc_feature 9..10: note = DNA
  misc_feature 11..100: note = RNA
  source 1..100: mol_type = other DNA

SEQ ID NO: 65 - DNA, 100 bp, synthetic construct; identical to SEQ ID NO: 41
  misc_feature 1..4: note = RNA
  misc_feature 5..6: note = DNA
  misc_feature 7..8: note = RNA
  misc_feature 9..10: note = DNA
  misc_feature 11..100: note = RNA
  source 1..100: mol_type = other DNA

SEQ ID NO: 66 - DNA, 100 bp, synthetic construct; identical to SEQ ID NO: 41
  misc_feature 1..2: note = RNA
  misc_feature 3..5: note = DNA
  misc_feature 6..8: note = RNA
  misc_feature 9..10: note = DNA
  misc_feature 11..100: note = RNA
  source 1..100: mol_type = other DNA

SEQ ID NO: 67 - DNA, 100 bp, synthetic construct; identical to SEQ ID NO: 41
  misc_feature 1..3: note = RNA
  misc_feature 4..6: note = DNA
  misc_feature 7..8: note = RNA
  misc_feature 9..10: note = DNA
  misc_feature 11..100: note = RNA
  source 1..100: mol_type = other DNA

SEQ ID NO: 68 - DNA, 100 bp, synthetic construct; identical to SEQ ID NO: 41
  misc_feature 1..2: note = RNA
  misc_feature 3..6: note = DNA
  misc_feature 7..8: note = RNA
  misc_feature 9..10: note = DNA
  misc_feature 11..100: note = RNA
  source 1..100: mol_type = other DNA

SEQ ID NO: 69 - RNA, 100 nt, synthetic construct; identical to SEQ ID NO: 40
  source 1..100: mol_type = other RNA
  modified_base 1..3: mod_base = OTHER; note = 2'O-methyl group
  modified_base 97..99: mod_base = OTHER; note = 2'O-methyl group
  modified_base 1..3: mod_base = OTHER; note = 2'-ribo 3'-phosphorothioate linkage
  modified_base 97..99: mod_base = OTHER; note = 2'-ribo 3' phosphorothioate linkage

SEQ ID NO: 70 - RNA, 100 nt, synthetic construct; identical to SEQ ID NO: 1
  source 1..100: mol_type = other RNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 71 - RNA, 100 nt, synthetic construct; identical to SEQ ID NO: 3
  source 1..100: mol_type = other RNA
  modified_base 1..3: mod_base = OTHER; note = 2'ribo-3'phosphorothioate linkage
  modified_base 97..99: mod_base = OTHER; note = 2'ribo-3'phosphorothioate linkage
  misc_feature 1..3: note = 2'O-methyl group
  modified_base 97..99: mod_base = OTHER; note = 2'O-methyl group

SEQ ID NO: 72 - DNA, 100 bp, synthetic construct; identical to SEQ ID NO: 41
  misc_feature 1..2: note = RNA
  misc_feature 3: note = DNA
  misc_feature 4..100: note = RNA
  source 1..100: mol_type = other DNA
  modified_base 1..3: mod_base = OTHER; note = 2'O-methyl group
  modified_base 97..99: mod_base = OTHER; note = 2'O-methyl group
  modified_base 1..3: mod_base = OTHER; note = 2'ribo-3'phosphorothioate linkage
  modified_base 97..99: mod_base = OTHER; note = 2'ribo-3'phosphorothioate linkage

SEQ ID NO: 73 - DNA, 100 bp, synthetic construct; identical to SEQ ID NO: 41
  misc_feature 1..3: note = RNA
  misc_feature 4: note = DNA
  misc_feature 5..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 74 - DNA, 100 bp, synthetic construct; identical to SEQ ID NO: 41
  misc_feature 1..4: note = RNA
  misc_feature 5: note = DNA
  misc_feature 6..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 75 - DNA, 100 bp, synthetic construct; identical to SEQ ID NO: 41
  misc_feature 1..5: note = RNA
  misc_feature 6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 76 - DNA, 100 bp, synthetic construct; identical to SEQ ID NO: 41
  misc_feature 1..6: note = RNA
  misc_feature 7: note = DNA
  misc_feature 8..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 77 - DNA, 100 bp, synthetic construct; identical to SEQ ID NO: 41
  misc_feature 1..7: note = RNA
  misc_feature 8: note = DNA
  misc_feature 9..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 78 - DNA, 100 bp, synthetic construct; identical to SEQ ID NO: 41
  misc_feature 1..8: note = RNA
  misc_feature 9: note = DNA
  misc_feature 10..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 79 - DNA, 100 bp, synthetic construct; identical to SEQ ID NO: 41
  misc_feature 1..9: note = RNA
  misc_feature 10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 80 - DNA, 100 bp, synthetic construct; identical to SEQ ID NO: 41
  misc_feature 1..2: note = RNA
  misc_feature 3..4: note = DNA
  misc_feature 5..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 81 - DNA, 100 bp, synthetic construct; identical to SEQ ID NO: 41
  misc_feature 1..3: note = RNA
  misc_feature 4..5: note = DNA
  misc_feature 6..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 82 - DNA, 100 bp, synthetic construct; identical to SEQ ID NO: 41
  misc_feature 1..4: note = RNA
  misc_feature 5..6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 83 - DNA, 100 bp, synthetic construct; identical to SEQ ID NO: 41
  misc_feature 1..5: note = RNA
  misc_feature 6..7: note = DNA
  misc_feature 8..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 84 - DNA, 100 bp, synthetic construct; identical to SEQ ID NO: 41
  misc_feature 1..6: note = RNA
  misc_feature 7..8: note = RNA
  misc_feature 9..100: note = DNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 85 - DNA, 100 bp, synthetic construct; identical to SEQ ID NO: 41
  misc_feature 1..7: note = RNA
  misc_feature 8..9: note = DNA
  misc_feature 10..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 86 - DNA, 100 bp, synthetic construct; identical to SEQ ID NO: 41
  misc_feature 1..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 87 - DNA, 100 bp, synthetic construct; identical to SEQ ID NO: 41
  misc_feature 1..2: note = RNA
  misc_feature 3..5: note = DNA
  misc_feature 6..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 88 - DNA, 100 bp, synthetic construct; identical to SEQ ID NO: 41
  misc_feature 1..3: note = RNA
  misc_feature 4..6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 89 - DNA, 100 bp, synthetic construct; identical to SEQ ID NO: 41
  misc_feature 1..4: note = RNA
  misc_feature 5..7: note = DNA
  misc_feature 8..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 90 - DNA, 100 bp, synthetic construct; identical to SEQ ID NO: 41
  misc_feature 1..5: note = RNA
  misc_feature 6..8: note = DNA
  misc_feature 9..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 91 - DNA, 100 bp, synthetic construct; identical to SEQ ID NO: 41
  misc_feature 1..6: note = RNA
  misc_feature 7..9: note = DNA
  misc_feature 10..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 92 - DNA, 100 bp, synthetic construct; identical to SEQ ID NO: 41
  misc_feature 1..7: note = RNA
  misc_feature 8..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 93 - DNA, 100 bp, synthetic construct; identical to SEQ ID NO: 41
  misc_feature 1..2: note = RNA
  misc_feature 3..6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 94 - DNA, 100 bp, synthetic construct; identical to SEQ ID NO: 41
  misc_feature 1..2: note = RNA
  misc_feature 3..4: note = DNA
  misc_feature 5..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 95 - DNA, 100 bp, synthetic construct; identical to SEQ ID NO: 41
  misc_feature 1..3: note = RNA
  misc_feature 4..5: note = DNA
  misc_feature 6..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 96 - DNA, 100 bp, synthetic construct; identical to SEQ ID NO: 41
  misc_feature 1..4: note = RNA
  misc_feature 5..6: note = DNA
  misc_feature 7..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 97 - DNA, 100 bp, synthetic construct; identical to SEQ ID NO: 41
  misc_feature 1..2: note = RNA
  misc_feature 3..5: note = DNA
  misc_feature 6..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 98 - DNA, 100 bp, synthetic construct; identical to SEQ ID NO: 41
  misc_feature 1..3: note = RNA
  misc_feature 4..6: note = DNA
  misc_feature 7..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 99 - DNA, 100 bp, synthetic construct; identical to SEQ ID NO: 41
  misc_feature 1..2: note = RNA
  misc_feature 3..6: note = DNA
  misc_feature 7..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 97..99: mod_base = OTHER; note = 2'ribo-3'phosphorothioate linkage
  modified_base 1..3: mod_base = OTHER; note = 2'O-methyl group
  modified_base 97..99: mod_base = OTHER; note = 2'O-methyl group

SEQ ID NO: 100 - RNA, 100 nt, synthetic construct; identical to SEQ ID NO: 40
  source 1..100: mol_type = other RNA
  modified_base 1..3: mod_base = OTHER; note = 2'O-methyl group
  modified_base 96..99: mod_base = OTHER; note = 2'ribo-3'phosphorothioate linkage
  modified_base 1..3: mod_base = OTHER; note = 2'ribo-3'phosphorothioate linkage
  modified_base 68..100: mod_base = OTHER; note = 2'O-methyl group
  modified_base 21..31: mod_base = OTHER; note = 2'O-methyl group

SEQ ID NO: 101 - RNA, 100 nt, synthetic construct; identical to SEQ ID NO: 1
  source 1..100: mol_type = other RNA
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group
  modified_base 1..3: mod_base = OTHER; note = 2'ribo-3'phosphorothioate linkage
  modified_base 96..99: mod_base = OTHER; note = 2'ribo-3'phosphorothioate linkage

SEQ ID NO: 102 - RNA, 100 nt, synthetic construct; identical to SEQ ID NO: 3
  source 1..100: mol_type = other RNA
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group
  modified_base 1..3: mod_base = OTHER; note = 2'ribo-3'phosphorothioate linkage
  modified_base 96..99: mod_base = OTHER; note = 2'ribo-3'phosphorothioate linkage

SEQ ID NO: 103 - DNA, 100 bp, synthetic construct; identical to SEQ ID NO: 41
  misc_feature 1..2: note = RNA
  misc_feature 3: note = DNA
  misc_feature 4..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 104 - DNA, 100 bp, synthetic construct; identical to SEQ ID NO: 41
  misc_feature 1..3: note = RNA
  misc_feature 4: note = DNA
  misc_feature 5..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 105 - DNA, 100 bp, synthetic construct; identical to SEQ ID NO: 41
  misc_feature 1..4: note = RNA
  misc_feature 5: note = DNA
  misc_feature 6..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 106 - DNA, 100 bp, synthetic construct; identical to SEQ ID NO: 41
  misc_feature 1..5: note = RNA
  misc_feature 6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 107 - DNA, 100 bp, synthetic construct; identical to SEQ ID NO: 41
  misc_feature 1..6: note = RNA
  misc_feature 7: note = DNA
  misc_feature 8..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 108 - DNA, 100 bp, synthetic construct; identical to SEQ ID NO: 41
  misc_feature 1..7: note = RNA
  misc_feature 8: note = DNA
  misc_feature 9..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 109 - DNA, 100 bp, synthetic construct; identical to SEQ ID NO: 41
  misc_feature 1..8: note = RNA
  misc_feature 9: note = DNA
  misc_feature 10..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 110 - DNA, 100 bp, synthetic construct; identical to SEQ ID NO: 41
  misc_feature 1..9: note = RNA
  misc_feature 10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 111 - DNA, 100 bp, synthetic construct; identical to SEQ ID NO: 41
  misc_feature 1..2: note = RNA
  misc_feature 3..4: note = DNA
  misc_feature 5..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 112 - DNA, 100 bp, synthetic construct; identical to SEQ ID NO: 41
  misc_feature 1..3: note = RNA
  misc_feature 4..5: note = DNA
  misc_feature 6..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 113 - DNA, 100 bp, synthetic construct; identical to SEQ ID NO: 41
  misc_feature 1..4: note = RNA
  misc_feature 5..6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 114 - DNA, 100 bp, synthetic construct; identical to SEQ ID NO: 41
  misc_feature 1..5: note = RNA
  misc_feature 6..7: note = DNA
  misc_feature 8..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 115 - DNA, 100 bp, synthetic construct; identical to SEQ ID NO: 41
  misc_feature 1..6: note = RNA
  misc_feature 7..8: note = DNA
  misc_feature 9..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 116 - DNA, 100 bp, synthetic construct; identical to SEQ ID NO: 41
  misc_feature 1..7: note = RNA
  misc_feature 8..9: note = DNA
  misc_feature 10..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 117 - DNA, 100 bp, synthetic construct; identical to SEQ ID NO: 41
  misc_feature 1..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 118 - DNA, 100 bp, synthetic construct; identical to SEQ ID NO: 41
  misc_feature 1..2: note = RNA
  misc_feature 3..5: note = DNA
  misc_feature 6..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 119 - DNA, 100 bp, synthetic construct; identical to SEQ ID NO: 41
  misc_feature 1..3: note = RNA
  misc_feature 4..6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 120 - DNA, 100 bp, synthetic construct; identical to SEQ ID NO: 41
  misc_feature 1..4: note = RNA
  misc_feature 5..7: note = DNA
  misc_feature 8..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 121 - DNA, 100 bp, synthetic construct; identical to SEQ ID NO: 41
  misc_feature 1..5: note = RNA
  misc_feature 6..8: note = DNA
  misc_feature 9..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 122 - DNA, 100 bp, synthetic construct; identical to SEQ ID NO: 41
  misc_feature 1..6: note = RNA
  misc_feature 7..9: note = DNA
  misc_feature 10..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 123 - DNA, 100 bp, synthetic construct; identical to SEQ ID NO: 41
  misc_feature 1..7: note = RNA
  misc_feature 8..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 124 - DNA, 100 bp, synthetic construct; identical to SEQ ID NO: 41
  misc_feature 1..2: note = RNA
  misc_feature 3..6: note = DNA
  misc_feature 7..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 125 - DNA, 100 bp, synthetic construct; identical to SEQ ID NO: 41
  misc_feature 1..2: note = RNA
  misc_feature 3..4: note = DNA
  misc_feature 5..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 126 - DNA, 100 bp, synthetic construct; identical to SEQ ID NO: 41
  misc_feature 1..3: note = RNA
  misc_feature 4..5: note = DNA
  misc_feature 6..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 127 - DNA, 100 bp, synthetic construct; identical to SEQ ID NO: 41
  misc_feature 1..4: note = RNA
  misc_feature 5..6: note = DNA
  misc_feature 7..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 128 - DNA, 100 bp, synthetic construct; identical to SEQ ID NO: 41
  misc_feature 1..2: note = RNA
  misc_feature 3..5: note = DNA
  misc_feature 6..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 129 - DNA, 100 bp, synthetic construct; identical to SEQ ID NO: 41
  misc_feature 1..3: note = RNA
  misc_feature 4..6: note = DNA
  misc_feature 7..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group

SEQ ID NO: 130 - DNA, 100 bp, synthetic construct; identical to SEQ ID NO: 41
  misc_feature 1..2: note = RNA
  misc_feature 3..6: note = DNA
  misc_feature 7..8: note = RNA
  misc_feature 9..10: note = DNA
  misc_feature 11..100: note = RNA
  source 1..100: mol_type = other DNA
  modified_base 1..3: mod_base = OTHER; note = 2'ribo-3'phosphorothioate linkage
  modified_base 96..99: mod_base = OTHER; note = 2'ribo-3'phosphorothioate linkage
  modified_base 1..3: mod_base = OTHER; note = 2'O-methyl group
  modified_base 21..31: mod_base = OTHER; note = 2'O-methyl group
  modified_base 68..100: mod_base = OTHER; note = 2'O-methyl group